(19) United States
(12) Patent Application Publication
(10) Pub. No.: US 2010/0069410 A1
(43) Pub. Date: Mar. 18, 2010

(54) ACYCLOVIR FORMULATIONS

(75) Inventors: Shingai Majuru, Brewster, NY (US); Moses Oyewumi, Yorktown Heights, NY (US)

Correspondence Address: Emisphere Technologies, Inc, 240 Cedar Knolls Road, Suite # 200, Cedar Knolls, NJ, 07927, US

(73) Assignee: Emisphere Technologies Inc., Cedar Knolls, NJ (US)

(21) Appl. No.: 12/516,798

(22) PCT Filed: Nov. 30, 2007
(86) PCT No.: PCT/US2007/086045

§ 371 Date: May 28, 2009

Related U.S. Application Data
(60) Provisional application No. 60/868,316, filed on Dec. 1, 2006.

(51) Int. Cl. A61K 31/522; A61P 31/22

(52) U.S. Cl. 514/263.38

(57) ABSTRACT

The present invention relates to acyclovir formulations having improved bioavailability resulting in better efficacy and/or requiring less frequent administration.

### FIELD OF THE INVENTION

The present invention relates to pharmaceutical formulations of acyclovir and delivery agent compounds providing increased acyclovir bioavailability.

### BACKGROUND OF THE INVENTION

Acyclovir (9-((2-hydroxyethoxy)methyl)guanine) is an antiviral which inhibits human herpes viruses, including herpes simplex types 1 (HSV-1) and 2 (HSV-2), varicella zoster, Epstein-Barr virus (EBV) and cytomegalovirus (CMV). The inhibitory activity of acyclovir is highly selective for these viruses. O'Brien and Campoli-Richards, Drugs, 37:233-309 (1989). The chemical composition of acyclovir is reported in Shaffer, et al. (J. Med. Chem. 14:367 (1971)), U.S. Pat. No. 4,199,574, and UK Patent Specification No. 1,523,865, all of which are hereby incorporated by reference.

Acyclovir has been demonstrated to be a potent antiviral agent, particularly against herpes viruses. Shaffer, et al. Nature 272:583-585 (1978). Acyclovir has also been demonstrated to effectively suppress reactivated or newly acquired viral diseases such as genital herpes simplex, shingles, and varicella-zoster, as well as acute varicella-zoster infections. Balfour, J. Med. Virology, S1:74-81 (1993). Morbidity and mortality from viral disease have been reduced by pre- and postoperative prophylaxis with long-term (>6 months) oral acyclovir therapy. Prentice et al., Lancet 343:749-753 (1994). Concurrent acyclovir and AZT (azidothymidine) therapy has extended the survival of AIDS patients by one year when acyclovir therapy was begun at time of diagnosis. Stein, et al., Ann. Intern. Med. 121:100-108 (1994). Additionally, acyclovir therapy for acute varicella-zoster disease reduces fever, chronic pain, and the progression of rash and accelerates cutaneous healing.

Other uses include, but are not limited to, mucocutaneous, ocular, and systemic herpes simplex infections (HSV), including in human immunodeficiency virus (HIV)-infected individuals. It is also useful to treat HSV encephalitis, neonatal HSV infections, and genital herpes (first episode, recurrent and suppressive therapy for recurrent infections). Further, acyclovir is effective therapy for varicella-zoster infections, herpes zoster (shingles, zoster), cytomegalovirus infections, infections and disorders associated with Epstein-Barr virus, and the Center for Disease Control states that oral acyclovir may be used in pregnant women. These and other uses are found in AHFS Drug Information, American Society of Health System Pharmacists, Bethesda, Md., 2005, which is incorporated by reference herein.

Acyclovir, is currently marketed as capsules, tablets and suspension for oral administration. Previously, orally administered acyclovir is slowly and erratically absorbed with a reported oral bioavailability of 15-30%. Barnhart (ed.), Physicians' Desk Reference, Oradell, N.J.: Medical Economics Data (1994). Over half the dose of the currently marketed formulation is recovered in the feces. Schaeffer et al., Nature, 272:583-585 (1978), Failure to respond to acyclovir therapy may arise from an inadequate dose (frequency of dose or total daily dose); patient noncompliance; malabsorption in the intestine; or resistant viral strains. Mindel, J. Med. Virology, S1:39-44 (1993). The need for readily absorbed oral antiviral agents has been identified as imperative for treatment of viral diseases to both patient populations since long term IV treatment is restrictive and compliance with currently available oral acyclovir formulations is difficult. Katlama, J. Med. Virology S1:128-133 (1993). An acyclovir preparation for oral delivery which permitted lower dosing and less frequent administration would facilitate compliance.

Previous attempts have been made to improve the oral delivery of acyclovir. U.S. Pat. No. 5,629,016, which is hereby incorporated by reference, discloses water dispersible tablets containing acyclovir which facilitates the ingestion of large doses (i.e. up to 800 mg) of acyclovir. The tablets, however, do not improve the bioavailability of the acyclovir.

U.S. Pat. No. 5,883,103 discloses a microemulsion system for the oral delivery of acyclovir. The system includes a water-in-oil emulsion with acyclovir dispersed in aqueous phase droplets. The droplets have an average droplet size of 20-40 nanometers and are uniformly dispersed in the continuous oil phase.

Although, previous attempts have been made to improve the delivery and bioavailability of acyclovir, these attempts have had limited success. Therefore, there is a need for oral acyclovir formulations having increased bioavailability.

### SUMMARY OF THE INVENTION

The present application provides acyclovir formulations comprising a delivery agent compound (e.g. SNAC) and one more additives that have improved acyclovir bioavailability.

Compositions of the present invention seek to provide enhanced solubility for acylovir, which is a hydrophobic compound. In one embodiment, the acyclovir is completely dissolved (or substantially completely dissolved) within one hour of contact with an aqueous medium (e.g. simulated gastric fluid or simulated intestinal fluid). The acyclovir formulations may further contain a disintegrant and/or a wetting agent to further increase the solubility of acyclovir in the aqueous medium.

Other compositions of the present invention facilitate simultaneous delivery of the delivery agent compound (e.g. SNAC) and acylovir to the site of absorption and/or upon contact with an aqueous medium.

Another embodiment of the present application provides a method of treating herpes viruses (e.g. HSV-1 and/or HSV-2) comprising administering one or more acyclovir formulations of the present invention to obtain Cₘₐₓ and/or AUC_{0-inf} values substantially equivalent to those for 1000 mg of valacyclovir dosage forms approved under NDA No. 20550.

### BRIEF DESCRIPTION OF THE DRAWINGS

FIG. 1 shows plasma acyclovir concentrations after dosing acyclovir/SNAC (240 mg/240 mg) formulation in beagles.

FIG. 2 shows mean plasma acyclovir concentrations in four beagles after dosing Acyclovir/SNAC (240 mg/240 mg) tablet formulation.

FIG. 3 shows plasma acyclovir concentrations in beagles after dosing acyclovir tablets with micronized SNAC.

FIG. 4 shows a comparison between the mean plasma acyclovir concentrations in beagles after dosing formulations made with either micronized or un-micronized SNAC.

FIG. 5 shows plasma Acyclovir concentrations in beagles after dosing acyclovir/SNAC formulation made with (0.2% w/w) Carbapol 934P.

FIG. 6 shows a comparison between the mean plasma acyclovir concentrations in beagles after dosing formulations that are made with or without Carbopol 934P.

FIG. 7 shows plasma acyclovir concentrations in beagles after dosing acyclovir/SNAC formulation made with 0.8% w/w Carbopol 934P.

FIG. 8 shows mean plasma acyclovir levels in beagles after dosing acyclovir/SNAC formulation made with (0.8%) Carbopol 934P.

FIG. 9 shows plasma acyclovir concentrations in beagles after dosing acyclovir/SNAC (240 mg/240 mg) tablet formulation made with (5% w/w) low molecular weight gelatin.

FIG. 10 shows plasma acyclovir concentrations in beagles after dosing acyclovir/SNAC (240 mg/240 mg) tablet formulation made with (10% w/w) low molecular weight gelatin.

FIG. 11 shows a comparison between mean plasma acyclovir concentrations in beagles after dosing three different acyclovir/SNAC (240 mg/240 mg) formulations: (i) 5% gelatin; (ii) 10% gelatin and (iii) a formulation without gelatin.

FIG. 12 shows mean plasma acyclovir levels in beagles after dosing acyclovir/SNAC (80 mg/240 mg) tablet formulation.

FIG. 13 shows mean plasma acyclovir levels in beagles after dosing acyclovir/SNAC (80 mg/240 mg) tablet formulation, as compared to ZOVIRAX® tablets.

FIG. 14 shows mean plasma acyclovir levels in beagles after dosing acyclovir/SNAC (80 mg/240 mg) formulation made with anhydrous dibasic calcium phosphate (Emcompress).

FIG. 15 shows mean plasma acyclovir levels in beagles after dosing acyclovir/SNAC tablets made with starch paste as the granulation agent.

FIG. 16 shows mean plasma acyclovir levels in beagles after dosing acyclovir/SNAC (240 mg/240 mg) formulation made with 0.5% Povidone K 90

FIG. 17 shows mean plasma acyclovir levels in beagles after dosing acyclovir/SNAC (240 mg/240 mg) formulation made with 2% w/w Povidone K90.

FIG. 18 shows mean plasma acyclovir levels in beagles after dosing acyclovir/SNAC (400 mg/240 mg) tablet formulation.

FIG. 19 shows mean plasma acyclovir levels in beagles after dosing acyclovir/SNAC (240 mg/80 mg) formulation.

FIG. 20 shows mean plasma acyclovir levels in beagles after dosing acyclovir/SNAC (240 mg/240 mg) formulation made with tween 80.

FIG. 21 shows plasma acyclovir concentrations in beagles after dosing acyclovir/SNAC (240 mg/240 mg) formulation comprising gelatin and sodium lauryl sulfate.

FIG. 22 shows mean plasma acyclovir levels in beagles after dosing acyclovir/SNAC (240 mg/240 mg) formulation comprising gelatin and sodium lauryl sulfate.

FIG. 23 shows mean plasma acyclovir levels in beagles after dosing acyclovir/SNAC (240 mg/240 mg) formulation made with lecithin.

FIG. 24 shows mean plasma acyclovir levels in beagles after dosing acyclovir/SNAC (80 mg/240 mg) capsule formulation.

FIG. 25 shows plasma Acyclovir concentrations in beagles after dosing Acyclovir/SNAC (240 mg/240 mg) formulation made with emulsifying solvents.

FIG. 26 shows mean plasma acyclovir levels in beagles after dosing acyclovir/SNAC (240 mg/240 mg) formulation made with emulsifying solvents.

FIG. 27 shows plasma acyclovir concentrations in beagles after dosing acyclovir/SNAC formulation made with emulsifying solvents.

FIG. 28 shows mean plasma acyclovir levels in beagles after dosing acyclovir/SNAC formulation made with emulsifying solvents.

FIG. 29 shows plasma acyclovir concentrations in beagles after dosing acyclovir/SNAC formulation made with potassium aluminum sulfate.

FIG. 30 shows mean plasma acyclovir levels in beagles after dosing another acyclovir/SNAC formulation made with potassium aluminum sulfate.

FIG. 31 shows plasma acyclovir concentrations in beagles after dosing acyclvoir/SNAC formulation made with lecithin and polysorbate 80.

FIG. 32 shows mean plasma acyclovir levels in beagles after dosing acyclovir/SNAC formulation made with lecithin and polysorbate 80

FIG. 33 shows plasma acyclovir concentrations in beagles after dosing acyclovir/SNAC (240 mg/240 mg) formulation made with gelatin and lecithin.

FIG. 34 shows mean plasma acyclovir levels in beagles after dosing acyclovir/SNAC (240 mg/240 mg) formulation made with gelatin and lecithin.

FIG. 35 shows plasma acyclovir concentrations in beagles after dosing acyclovir/SNAC (360 mg/360 mg) formulation comprising lecithin.

FIG. 36 shows mean plasma acyclovir levels in beagles after dosing acyclovir/SNAC (360 mg/360 mg) formulation comprising lecithin.

FIG. 37 shows plasma acyclovir concentrations in beagles after dosing acyclovir/SNAC (360 mg/360 mg) formulation comprising gelatin and sodium lauryl sulfate.

FIG. 38 shows mean plasma acyclovir levels in beagles after dosing acyclovir/SNAC (360 mg/360 mg) formulation made with gelatin and sodium lauryl sulfate.

FIG. 39 shows plasma acyclovir concentrations in beagles after dosing acyclovir/SNAC (360 mg/360 mg formulation made with polysorbate 80.

FIG. 40 shows mean plasma acyclovir levels after dosing acyclovir/SNAC (360 mg/360 mg) formulation made with polysorbate 80.

FIG. 41 shows plasma acyclovir concentrations in beagles after dosing acyclovir/SNAC (360 mg/360 mg) formulation made with Gelucire.

FIG. 42 shows mean plasma acyclovir levels in beagles after dosing acyclovir/SNAC (360 mg/360 mg) formulation made with Gelucire.

FIG. 43 shows plasma acyclovir concentrations in beagles after dosing acyclovir/SNAC formulation made with PEG 300 and Capmul.

FIG. 44 shows mean plasma acyclovir levels in beagles after dosing acyclovir/SNAC (300 mg/300 mg) formulation made with PEG 300 and Capmul.

FIG. 45 shows plasma Acyclovir concentrations in beagles after dosing a commercial acyclovir (ZOVIRAX®) 400 mg tablets.

FIG. 46 shows mean plasma Acyclovir levels in beagles after dosing commercial acyclovir (ZOVIRAX®) 400 mg tablets.

FIG. 47 shows average plasma acyclovir concentrations based on the clinical study in Example 30—without logarithmic transformation.

FIG. 48 shows average plasma acyclovir concentrations based on the clinical study in Example 30—with logarithmic transformation.

### DETAILED DESCRIPTION OF THE INVENTION

### Definitions

The term “hydrate” as used herein includes, but is not limited to, (i) a substance containing water combined in the molecular form and (ii) a crystalline substance containing one or more molecules of water of crystallization or a crystalline material containing free water.

The term “solvate” as used herein includes, but is not limited to, a molecular or ionic complex of molecules or ions of a solvent with molecules or ions of the delivery agent compound or salt thereof, or hydrate or solvate thereof.

The term “delivery agent” or “delivery agent compound” refers to any of the delivery agent compounds disclosed herein.

The term “SNAC” refers to N-(8-[2-hydroxybenzoyl]-amino)caprylic acid, and pharmaceutically acceptable salts thereof. The term “monosodium salt of SNAC” refers to the monosodium salt of N-(8-[2-hydroxybenzoyl]-amino)caprylic acid. Unless otherwise noted, the term “SNAC” refers to all forms of SNAC, including all amorphous and polymorphic forms of SNAC, such as SNAC trihydrate and those described in U.S. Ser. Nos. 60/619,418 and 60/569,476, both of which are hereby incorporated by reference. The term “SNAC trihydrate” as used herein refers to a crystalline form of SNAC in which three molecules of water are associated with each molecule of SNAC. SNAC can be prepared by the procedures described in U.S. Pat. No. 5,650,386 and International Publication Nos. WO00/46182 and WO00/59863.

The term “SNAD” refers to N-(10-[2-hydroxybenzoyl]-amino)decanoic acid, and pharmaceutically acceptable salts thereof. The term “monosodium salt of SNAD” refers to the monosodium salt of N-(10-[2-hydroxybenzoyl]-amino)decanoic acid and the term “disodium salt of SNAD” refers to the disodium salt of N-(10-[2-hydroxybenzoyl]-amino)decanoic acid.

The term “4-CNAB” refers to 4-[(4-chloro-2-hydroxy-benzoyl)amino]butanoic acid (also known as 4-[(2-hydroxy-4-chlorobenzoyl)amino]butanoate), and pharmaceutically acceptable salts thereof. The term “monosodium salt of 4-CNAB” refers to the monosodiuim salt of 4-[(4-chloro-2-hydroxy-benzoyl)amino]butanoic acid.

An “effective amount of acyclovir” is an amount of acyclovir which is effective to treat or prevent a condition in a living organism to whom it is administered over some period of time, e.g., provides a therapeutic effect during a desired dosing interval.

An “effective amount of delivery agent” is an amount of the delivery agent which enables and/or facilitates the absorption of a desired amount of acyclovir via any route of administration (such as those discussed in this application including, but not limited to, the oral (e.g., across a biological membrane in the gastrointestinal tract), nasal, pulmonary, dermal, buccal, vaginal, and/or ocular route).

The term “mean”, when preceding a pharmacokinetic value (e.g., mean peak) represents the arithmetic mean value of the pharmacokinetic value unless otherwise specified.

As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

The term “about” generally means within 10%, preferably within 5%, and more preferably within 1% of a given value or range.

The terms “alkyl” and “alkenyl” as used herein include linear and branched alkyl and alkenyl substituents, respectively.

The phrase “pharmaceutically acceptable” refers to additives or compositions that are physiologically tolerable and do not typically produce an allergic or similar untoward reaction, such as gastric upset, dizziness and the like, when administered to a mammal.

By “condition or disorder caused by a virus” is meant any condition or disorder in an animal that is either caused by, complicated by, or aggravated by a virus. Such conditions or disorders include, but are not limited to, those caused by viruses of the herpes family, for example, herpes simplex 1 and 2 viruses (HSV 1, HSV 2), varicella zoster virus (VZV), cytomegalovirus (CMV), Epstein-Barr virus (EBV), and other herpes virus infections (e.g. feline herpes virus infections).

As used herein, the term “treat” includes one or more of the following:

(a) arresting, delaying the onset (i.e., the period prior to clinical manifestation of a disorder) and/or reducing the risk of developing or worsening a disorder;

(b) relieving or alleviating at least one symptom of a disorder in a mammal, including for example, hypercalcemia; or

(c) relieving or alleviating the intensity and/or duration of a manifestation of a disorder experienced by a mammal including, but not limited to, those which are in response to a given stimulus (e.g., pressure, tissue injury or cold temperature). The turn “treat” also includes prophylactically preventing, curing, healing, alleviating, relieving, altering, remedying, ameliorating, improving, or affecting a condition (e.g., a disease), the symptoms of the condition, or the predisposition toward the condition.

As used herein, the term “dissolve” or “dissolved” refers to the process in which solid particles mix molecule by molecule with a liquid or semi-solid solvent (e.g. a gel) and appear to become part of the liquid or semi-solid solvent.

An example of simulated gastric fluid (SGF) is SGF of pH 1.2 prepared as per the USP NF 26 guidelines. More particularly, 2 g sodium chloride and 3.2 g of pepsin may be weighed added to a suitable container, and deionized water may be added to reach one liter in volume. If necessary, the pH may be adjusted to 1.2 by addition of concentrated HCl or NaOH.

An example of simulated intestinal fluid (SIF) is SIF prepared as per the USP NF 26 guidelines. More particularly, SIF may be prepared by addition of 6.8 g monobasic potassium phosphate and 10 g of pancreatin into a suitable vessel, and deionized water may be added to reach a total volume of one liter. If necessary, the pH may be adjusted to 7.5 by addition of 0.2 N sodium hydroxide.

### Acyclovir

The term “acyclovir” refers to 9-(2-hydroxyethoxymethyl) guanine. Suitable salts (e.g., pharmaceutically acceptable salts) and esters of acyclovir are described in U.S. Pat. No. 4,199,574, which is hereby incorporated by reference, and include, but are not limited to, sodium acyclovir and acyclovir valerate. Acyclovir also forms acid addition salts, such as with hydrochloric, sulphuric, phosphoric, maleic, fumaric, citric, tartaric, lactic and acetic acid.

A synthesis of acyclovir is disclosed in U.S. Pat. No. 4,199,574, which is hereby incorporated by reference. Acyclovir is commercially available from GlaxoSmithKline (Research Triangle Park, N.C.) under the tradename ZOVIRAX®.

Any prodrug which is converted in vivo to 9-(2-hydroxyethoxymethyl) guanine can also be used. The term “prodrug” as used herein includes pharmaceutically acceptable salts of the drug. Acyclovir prodrugs include, substituted purines of the formula:

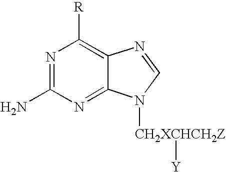

or salts thereof, wherein:

R is hydrogen, hydroxy, or amino;

X is oxygen or sulphur;

Y is hydrogen or hydroxymethyl; and

Z is —H, C₁₋₁₆ alkyl, or —OCOCH(R₁)NH₂, wherein R₁ is —CH[CH₃]₂.

Suitable acyclovir prodrugs, include but are not limited to, those described in U.S. Pat. Nos. 4,609,662, 4,758,572 and 4,957,924, all of which are hereby incorporated by reference. A non-limiting example of such a prodrug is 2-[(2-amino-1,6-dihydro-6-oxo-9H-purin-9-yl)methoxy]ethyl ester (valacyclovir) and its pharmaceutically acceptable salts. Valacyclovir is commercially available as its hydrochloride salt from GlaxoSmithKline (Research Triangle Park, N.C.) under the tradename Valtrex™.

Therapeutically effective amounts of a acyclovir for use in treatment of all conditions and disorders described herein, is an amount sufficient to suppress or alleviate conditions associated with the viral infection. As will be recognized by those in the field, an effective amount of therapeutic agent will vary with many factors including the potency of the acyclovir or salt, ester, or prodrug thereof, the age and weight of the patient, and the severity of the condition or disorder to be treated.

According to one embodiment, the acyclovir (or a salt, ester, prodrug thereof) is administered (e.g. peripherally) at a dose of about 0.1 to about 250 mg per kilogram of body weight of the recipient per day (mg/kg/day), about 1 to about 100 mg/kg/day, or about 5 to about 20 mg/kg/day (based on the weight of acyclovir). According to another embodiment, the dose is about 10 mg/kg/day. The desired dose may be administered either as a single or divided dose.

The acyclovir and delivery agent compound may be administered separately or together with one or more other active agents. For example, the acyclovir and delivery agent compound may be administered separately or together with compounds or compositions that exhibit antiviral activity, such as compounds used to treat retroviral infections (particularly HIV infections), e.g., 3′-azido-3′-deoxythymidine (AZT) and/or compounds or compositions that exhibit activity as ribonucleotide reductase inhibitors. Suitable ribonucleotide reductase inhibitors include, but are not limited to, thiocarbonohydrazone ribonucleotide reductase inhibitors, such as those disclosed in U.S. Pat. No. 5,393,883, which is hereby incorporated by reference.

### Delivery Agent Compounds

In one embodiment of the present invention, the delivery agent compound has the following structure, or a pharmaceutically acceptable salt thereof:

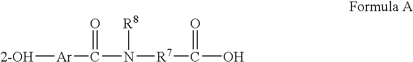

wherein

Ar is phenyl or naphthyl;

Ar is optionally substituted with one or more of —OH, halogen, C₁-C₄ alkyl, C₁-C₄ alkenyl, C₁-C₄ alkoxy or C₁-C₄ haloalkoxy;

R⁷ is C₄-C₂₀ alkyl, C₄-C₂₀ alkenyl, phenyl, naphthyl, (C₁-C₁₀ alkyl) phenyl, (C₁-C₁₀ alkenyl)phenyl, (C₁-C₁₀ alkyl) naphthyl, (C₁-C₁₀ alkenyl) naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl);

R⁸ is hydrogen, C₁ to C₄ alkyl, C₂ to C₄ alkenyl, C₁ to C₄ alkoxy, or C₁-C₄ haloalkoxy;

R⁷ is optionally substituted with C₁ to C₄ alkyl, C₂ to C₄ alkenyl, C₁ to C₄ alkoxy, C₁-C₄ haloalkoxy, —OH, —SH, —CO₂R⁹, or any combination thereof;

R⁹ is hydrogen, C₁ to C₄ alkyl, or C₂ to C₄ alkenyl; and

R⁷ is optionally interrupted by oxygen, nitrogen, sulfur or any combination thereof.

In one embodiment, the delivery agent compounds are not substituted with an amino group in the position alpha to the acid group.

According to one embodiment, R⁷ in Formula A is selected from C₈-C₂₀ alkyl, C₈-C₂₀ alkenyl, phenyl, naphthyl, (C₁-C₁₀ alkyl) phenyl, (C₁-C₁₀ alkenyl)phenyl, (C₁-C₁₀ alkyl) naphthyl, (C₁-C₁₀ alkenyl)naphthyl, phenyl(C₁-C₁₀ alkyl), phenyl(C₁-C₁₀ alkenyl), naphthyl(C₁-C₁₀ alkyl), and naphthyl(C₁-C₁₀ alkenyl).

According to another embodiment, R⁷ in Formula A is selected from C₈-C₂₀ alkyl, and C₈-C₂₀ alkenyl.

In another embodiment of the present invention, the delivery agent compound has the following structure, or a pharmaceutically acceptable salt thereof:

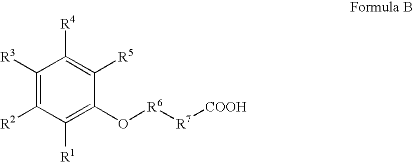

wherein

R¹, R², R³, and R⁴ are independently H, —OH, halogen, C₁-C₄ alkyl, C₂-C₄ alkenyl, C₁-C₄ alkoxy, —C(O)R⁸, —NO₂, —NR⁹R¹⁰, or —N+R⁹R¹⁰R¹¹(R¹²)⁻;

R⁵ is H, —OH, —NO₂, halogen, —CF₃, —NR⁹R¹⁰, —N⁺R¹⁴R¹⁵R¹⁶(R¹³)⁻, amide, C₁-C₁₂ alkoxy, C₁-C₁₂ alkyl, C₂-C₁₂ alkenyl, carbamate, carbonate, urea, or —C(O)R¹⁸;

R⁵ is optionally substituted with halogen, —OH, —SH, or —COOH;

R⁵ is optionally interrupted by O, N, S, or —C(O)—;

R⁶ is a C₁-C₁₂ alkylene, C₂-C₁₂ alkenylene, or arylene;

R⁶ is optionally substituted with a C₁-C₄ alkyl, C₂-C₄ alkenyl, C₁-C₄ alkoxy, —OH, —SH, halogen, —NH₂, or —CO₂R⁸;

R⁶ is optionally interrupted by O or N;

R⁷ is a bond or arylene;

R⁷ is optionally substituted with —OH, halogen, —C(O)CH₃, —NR¹⁰R¹¹, or —N⁺R¹⁰R¹¹R¹²(R¹³)⁻;

each occurrence of R⁸ is independently H, C₁-C₄ alkyl, C₂-C₄ alkenyl, or NH₂;

R⁹, R¹⁰, R¹¹, and R¹² independently H or C₁-C₁₀ alkyl;

R¹³ is a halide, hydroxide, sulfate, tetrafluoroborate, or phosphate;

R¹⁴, R¹⁵ and R¹⁶ are independently H, C₁-C₁₀ alkyl, C₁-C₁₀ alkyl substituted with —COOH, C₂-C₁₂ alkenyl, C₂-C₁₂ alkenyl substituted with —COOH, or —C(O)R¹⁷;

R¹⁷ is —OH, C₁-C₁₀ alkyl, or C₂-C₁₂ alkenyl; and

R¹⁸ is H, C₁-C₆ alkyl, —OH, —NR¹⁴R¹⁵, or N⁺R¹⁴R¹⁵R¹⁶(R¹³)⁻.

In one particular embodiment, when R¹, R², R³, R⁴, and R⁵ are H, and R⁷ is a bond then R⁶ is not a C₁-C₆, C₉ or C₁₀ alkyl.

In another embodiment, when R¹, R², R³, and R⁴ are H, R⁵ is —OH, and R⁷ is a bond then R⁶ is not a C₁-C₃ alkyl.

In yet another embodiment, when at least one of R¹, R², R³, and R⁴ is not H, R⁵ is —OH, and R⁷ is a bond, then R⁶ is not a C₁-C₄ alkyl.

In yet another embodiment, when R¹, R², and R³ are H, R⁴ is —OCH₃, R⁵ is —C(O)CH₃, and R⁶ is a bond then R⁷ is not a C₃ alkyl.

In yet another embodiment, when R¹, R², R⁴, and R⁵ are H, R³ is —OH, and R⁷ is a bond then R⁶ is not a methyl.

In yet another embodiment, R⁶ of Formula B is a C₈-C₁₂ alkylene, C₈-C₁₂ alkenylene, or arylene.

In yet another embodiment of the present invention, the delivery agent compound has the following structure or a pharmaceutically acceptable salt thereof:

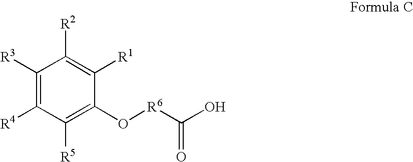

wherein

R¹, R², R³, R⁴ and R⁵ are independently H, —CN, —OH, —OCH₃, or halogen, at least one of R¹, R², R³, R⁴ and R⁵ being —CN; and

R⁶ is a C₁-C₁₂ linear or branched alkylene, a C₁-C₁₂ linear or branched alkenylene, a C₁-C₁₂ linear or branched arylene, an alkyl(arylene) or an aryl(alkylene).

According to one embodiment, when R¹ is —CN, R⁴ is H or —CN, and R², R³, and R⁵ are H, then R⁶ is not methylene ((CH₂)₁).

In another embodiment, R⁶ of Formula C is a C₉-C₁₂ linear or branched alkylene, a C₈-C₁₂ linear or branched alkenylene, an arylene, an alkyl(arylene) or an aryl(alkylene).

In yet another embodiment, R⁶ of Formula C is a C₈-C₁₂ linear or branched alkylene, a C₈-C₁₂ linear or branched alkenylene

Other suitable delivery agent compounds are disclosed in U.S. Pat. No. 6,627,228, which is hereby incorporated by reference.

In embodiments of the present invention, delivery agent compounds to be used in the topical composition along with the acyclovir compound include, but are not limited to, a polymeric delivery agent comprising a polymer conjugated to a modified amino acid or derivative thereof via a linkage group selected from the group consisting of —NHC(O)NH—, —C(O)NH—, —NHC(O)—, —OOC—, —COO—, —NHC(O)O—, —OC(O)NH—, —CH₂NH—, —NHCH₂—, —CH₂NHC(O)O—, —OC(O)NHCH₂—, —CH₂NHCOCH₂O—, —OCH₂C(O)NHCH₂—, —NHC(O)CH₂O—, —OCH₂C(O)NH—, —NH—, —O—, and carbon-carbon bond. In one embodiment, the polymeric delivery agent is not a polypeptide or polyamino acid. In another embodiment, the modified amino acid has the structure of formula A, B, or C. In one embodiment, the polymeric delivery agent includes a modified amino acid having the structure:

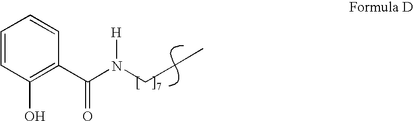

which is conjugated via a —COO group to a polymer having monomers derived from polyethylene glycol.

In one embodiment, the polymeric delivery agent is a modified amino acid having the structure of Formula D conjugated via a —COO group to a polymer having the structure:

—CH₂CH₂O(CH₂CH₂O)ₓCH₂CH₂O—Y,

wherein

x is from 1-14; and

Y is H or CH₃.

According to one embodiment, the polymeric delivery agent is compound having the structure of Formula D conjugated via a —COO group to a polymer having the structure:

—CH₂CH₂O(CH₂CH₂O)ₓCH₂CH₂O—Y,

wherein
x is 1-9; and
Y is CH₃ or H. For example, the polymeric delivery agent can be 8-(2-hydroxybenzoylamino)-octanoic acid 2-{2-[2-(2-{2-[2-(2-methoxyethoxy)ethoxy]ethoxy}-ethoxy)ethoxy]ethoxy}ethyl ester.

In one embodiment, the delivery agent compound is PEGylated SNAC with an average of about 6-9 or about 7-8 (e.g. 7.3) repeating ethylene oxide groups and having a molecular weight of about 500-800 (e.g. 600) daltons.

Delivery agent compounds of the present invention include compounds as shown below and pharmaceutically acceptable salts thereof:

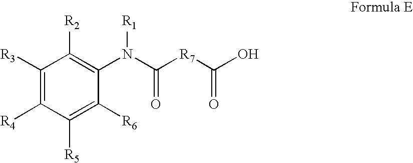

wherein:

R₁ is X (CH₂)ₘ—R₈, wherein m=0 or 1;

R₂-R₆ are independently selected from hydrogen, hydroxyl, halogen, C₁-C₄ alkyl, C₂-C₄alkenyl, C₂-C₄ alkynyl, C₁-C₄ alkoxy, and cyano;

R₇ is selected from C₁-C₁₀ alkyl, C₂-C₁₀ alkenyl, and C₂-C₁₀ alkynyl;

R₈ is selected from cyclopentyl, cyclohexyl and phenyl, wherein when R₈ is a phenyl, m=1; and

R₈ is optionally substituted with C₁-C₄ alkyl, C₁-C₄ alkoxy, halogen or hydroxyl, or a combination thereof.

Other delivery agent compounds of the present invention include those of the formula:

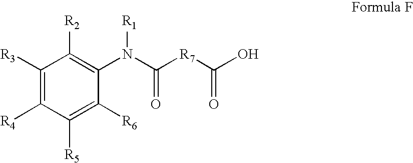

and pharmaceutically acceptable salts thereof, wherein:

R₁ is a C₁-C₆ alkyl, or C₂-C₆ alkenyl,

R₂-R₆ are independently chosen from the group consisting of hydrogen, hydroxyl, halogen, C₁-C₄ alkyl, C₂-C₄ alkenyl, C₂-C₄ alkynyl, C₁-C₄ alkoxy, and cyano, and

R₇ is selected from the group consisting of C₁-C₁₀ alkyl, C₂-C₁₀ alkenyl, and C₂-C₁₀ alkynyl.

Other delivery agent compounds of the present invention include those of the formula:

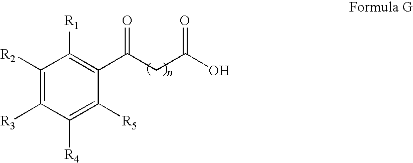

and pharmaceutically acceptable salts thereof, wherein

n=1 to 9, and

R₁ to R₅ are independently hydrogen, C₁ to C₄ alkyl, C₁ to C₄ alkoxy, C₂ to C₄ alkenyl, halogen, hydroxyl, —NH—C(O)—CH₃, or —O—C₆H₅.

Other delivery agent compounds of the present invention include those of the

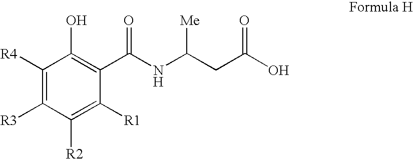

and pharmaceutically acceptable salts thereof, wherein

R₁ to R₄ are independently hydrogen, C₁ to C₄ alkyl, C₂ to C₄ alkenyl, halogen, C₁ to C₄ alkoxy, or hydroxyl.

Other delivery agent compounds of the present invention include those of the formula:

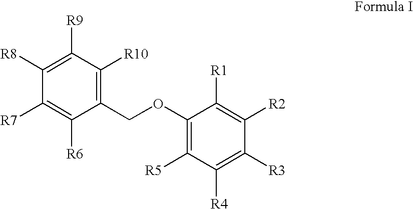

and pharmaceutically acceptable salts thereof, wherein

one of R1 to R5 has the generic structure

—(CH2)n-COOH

where n=0-6;

the remaining four members of R₁ to R₅ are independently hydrogen, C₁ to C₄ alkyl, C₂ to C₄ alkenyl, halogen, C₁ to C₄ alkoxy, or hydroxyl; and

R₆-R₁₀ are independently hydrogen, C₁ to C₄ alkyl, C₂ to C₄ alkenyl, halogen, C₁ to C₄ alkoxy, or hydroxyl.

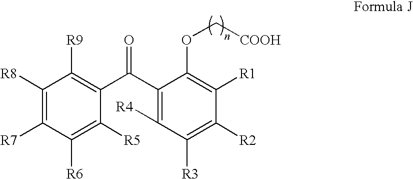

and pharmaceutically acceptable salts thereof, wherein

n=1 to 9; and R₁ to R₉ are independently hydrogen, C₁ to C₄ alkyl, C₂ to C₄ alkenyl, halogen, C₁ to C₄ alkoxy, or hydroxyl.

Other delivery agent compounds of the present invention include those of the formula:

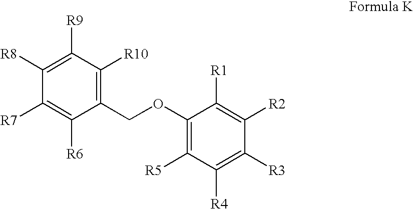

and pharmaceutically acceptable salts thereof, wherein

R₁-R₅ are independently hydrogen, C₁ to C₄ alkyl, C₂ to C₄ alkenyl, halogen, C₁ to C₄ alkoxy, hydroxyl, or —O—(CH2)n—COOH (where n is 1 to 12);

at least one of R₁ to R₅ has the generic structure

—O—(CH2)n-COOH

where n=1-12; and

R₆-R₁₀ are independently hydrogen, C₁ to C₄ alkyl, C₂ to C₄ alkenyl, halogen, C₁ to C₄ alkoxy, or hydroxyl. International Application Nos. PCT/US2005/017339 and PCT/US2005/017309, filed May 16, 2005 (Attorney Docket Nos. 01946/2201284-WO0 and 01946/2201285-WO0) and their priority documents, U.S. Provisional Application Nos. 60/576,088, filed Jun. 1, 2004, U.S. Provisional Application No. 60/576,397, filed Jun. 1, 2004, U.S. Provisional Application No. 60/576,105, filed Jun. 1, 2004, U.S. Provisional Application No. 60/571,090, filed May 14, 2004, U.S. Provisional Application No. 60/571,092, filed May 14, 2004, U.S. Provisional Application No. 60/571,195, filed May 14, 2004, U.S. Provisional Application No. 60/571,194, filed May 14, 2004, U.S. Provisional Application No. 60/571,093, filed May 14, 2004, U.S. Provisional Application No. 60/571,055, filed May 14, 2004, U.S. Provisional Application No. 60/571,151, filed May 14, 2004, U.S. Provisional Application No. 60/571,315, filed May 14, 2004, U.S. Provisional Application No. 60/571,144, filed May 14, 2004, and U.S. Provisional Application 60/571,089, filed May 14, 2004, are hereby incorporated by reference in their entirety.

The delivery agent compound may be any of those described in U.S. Pat. Nos. 6,699,467, 6,663,898, 6,693,208, 6,693,073, 6,693,898, 6,663,887, 6,646,162, 6,642,411, 6,627,228, 6,623,731, 6,610,329, 6,558,706, 6,525,020, 6,461,643, 6,461,545, 6,440,929, 6,428,780, 6,413,550, 6,399,798, 6,395,774, 6,391,303, 6,384,278, 6,375,983, 6,358,504, 6,346,242, 6,344,213, 6,331,318, 6,313,088, 6,245,359, 6,242,495, 6,221,367, 6,180,140, 6,100,298, 6,100,285, 6,099,856, 6,090,958, 6,084,112, 6,071,510, 6,060,513, 6,051,561, 6,051,258, 6,001,347, 5,990,166, 5,989,539, 5,976,569, 5,972,387, 5,965,121, 5,962,710, 5,958,451, 5,955,503, 5,939,381, 5,935,601, 5,879,681, 5,876,710, 5,866,536, 5,863,944, 5,840,340, 5,824,345, 5,820,881, 5,811,127, 5,804,688, 5,792,451, 5,776,888, 5,773,647, 5,766,633, 5,750,147, 5,714,167, 5,709,861, 5,693,338, 5,667,806, 5,650,386, 5,643,957, 5,629,020, 5,601,846, 5,578,323, 5,541,155, 5,540,939, 5,451,410, 5,447,728, 5,443,841, and 5,401,516; International Publication Nos. WO94/23767, WO95/11690, WO95/28920, WO95/28838, WO96/10396, WO96/09813, WO96/12473, WO97/36480, WO 2004/4104018, WO 2004080401, WO 2004062587, WO 2003/057650, WO 2003/057170, WO 2003/045331, WO 2003/045306, WO 2003/026582, WO 2002/100338, WO 2002/070438, WO 2002/069937, WO 02/20466, WO 02/19969, WO 02/16309, WO 02/15959, WO 02/02509, WO 01/92206, WO 01/70219, WO 01/51454, WO 01/44199, WO 01/34114, WO 01/32596, WO 01/32130, WO 00/07979, WO 00/06534, WO 00/06184, WO 00/59863, WO 00/59480, WO 00/50386, WO 00/48589, WO 00/47188, WO 00/46182, WO 00/40203, WO 99/16427, WO 98/50341, WO 98/49135, WO 98/34632, WO 98/25589, WO 98/21951, WO 97/47288, WO 97/31938, WO 97/10197, WO 96/40076, WO 96/40070, WO 96/39835, WO 96/33699, WO 96/30036, WO 96/21464, WO 96/12475, and WO 96/12474; and U.S. Published Application Nos. 20040110839, 20040106825, 20040068013, 20040062773, 20040022856, 20030235612, 20030232085, 20030225300, 20030198658, 20030133953, 20030078302, 20030072740, 20030045579, 20030012817, 20030008900, 20020155993, 20020127202, 20020120009, 20020119910, 20020102286, 20020065255, 20020052422, 20020040061, 20020028250, 20020013497, 20020001591, 20010039258, and 20010003001. Each of the above listed U.S. patents and U.S. and International published applications are herein incorporated by reference.

Non-limiting examples of delivery agent compounds include N-(8-[2-hydroxybenzoyl]-amino)caprylic acid, N-(10-[2-hydroxybenzoyl]-amino)decanoic acid, 8-(2-hydroxy-4-methoxybenzoylamino)octanoic acid, 8-(2,6-dihydroxybenzoylamino)octanoic acid, 8-(2-hydroxy-5-bromobenzoylamino)octanoic acid, 8-(2-hydroxy-5-chlorobenzoylamino)octanoic acid, 8-(2-hydroxy-5-iodobenzoylamino)octanoic acid, 8-(2-hydroxy-5-methylbenzoylamino)octanoic acid, 8-(2-hydroxy-5-fluorobenzoylamino)octanoic acid, 8-(2-hydroxy-5-methoxybenzoylamino)octanoic acid, 8-(3-hydroxyphenoxy)octanoic acid, 8-(4-hydroxyphenoxy)octanoic acid, 6-(2-cyanophenoxy)hexanoic acid, 8-(2-Hydroxyphenoxy)octyl-diethanolamine, 8-(4-hydroxyphenoxy)octanoate, 8-(4-hydroxyphenoxy)octanoate, 8-(2-hydroxy-4-methoxybenzoylamino)octanoic acid, 8-(2-hydroxy-5-methoxybenzoylamino)-octanoic acid., and salts thereof. Preferred salts include, but are not limited to, monosodium and disodium salts.

The delivery agent compounds may be in the form of the carboxylic acid or pharmaceutically acceptable salts thereof, such as sodium salts, and hydrates and solvates thereof. The salts may be mono- or multi-valent salts, such as monosodium salts and disodium salts. The delivery agent compounds may contain different counter ions chosen for example due to their effect on modifying the dissolution profile of the delivery agent compound.

The delivery agent compounds may be prepared by methods known in the art, such as those discussed in the aforementioned publications (e.g., International Publication Nos. WO 98/34632, WO 00/07979, WO 01/44199, WO 01/32596, WO 02/20466, and WO 03/045306). SNAC, SNAD, and the free acid and other salts thereof may be prepared by methods known in the art, such as those described in U.S. Pat. Nos. 5,650,386 and 5,866,536.

Salts of the delivery agent compounds of the present invention may be prepared by methods known in the art. For example, sodium salts may be prepared by dissolving the delivery agent compound in ethanol and adding aqueous sodium hydroxide.

The delivery agent compound may be purified by recrystallization or by fractionation on one or more solid chromatographic supports, alone or linked in tandem. Suitable recrystallization solvent systems include, but are not limited to, acetonitrile, methanol, and tetrahydrofuran. Fractionation may be performed on a suitable chromatographic support such as alumina, using methanol/n-propanol mixtures as the mobile phase; reverse phase chromatography using trifluoroacetic acid/acetonitrile mixtures as the mobile phase; and ion exchange chromatography using water or an appropriate buffer as the mobile phase. When anion exchange chromatography is performed, preferably a 0-500 mM sodium chloride gradient is employed.

### Delivery Systems

The composition of the present invention comprises one or more delivery agent compounds of the present invention and acyclovir. The delivery agent compound and acyclovir are typically mixed prior to administration to form an administration composition.

The administration compositions may be in the form of a liquid. The solution medium may be water, 25% aqueous propylene glycol, or phosphate buffer. Other dosing vehicles include polyethylene glycol. Dosing solutions may be prepared by mixing a solution of the delivery agent compound with a solution of the active agent, just prior to administration. Alternately, a solution of the delivery agent compound (or acyclovir) may be mixed with the solid form of acyclovir (or delivery agent compound). The delivery agent compound and acyclovir may also be mixed as dry powders. The delivery agent compound and acyclovir can also be admixed during the manufacturing process.

The dosing solutions may optionally contain additives such as phosphate buffer salts, citric acid, glycols, or other dispersing agents. Stabilizing additives may be incorporated into the solution, preferably at a concentration ranging between about 0.1 and 20% (w/v).

For example, the compositions useful in the invention can be provided as parenteral compositions (e.g., injection or infusion). According to one embodiment, the composition is suspended in an aqueous carrier, such as in an isotonic buffer solution at a pH of about 3.0 to about 8.0. Suitable buffers include, but are not limited to, sodium citrate-citric acid and sodium phosphate-phosphoric acid, and sodium acetate/acetic acid buffers.

The administration compositions may alternately be in the form of a solid, such as a tablet, capsule or particle, such as a powder or sachet. Solid dosage forms may be prepared by mixing the solid form of the compound with the solid form of acyclovir. Alternately, a solid may be obtained from a solution of compound and acyclovir by methods known in the art, such as freeze-drying (lyophilization), precipitation, crystallization and solid dispersion. Alternatively, the administration can be a semi-solid, in the form of a gel, paste, colloid, gelatin, emulsion, suspension and the like.

The administration compositions of the present invention may also include one or more enzyme inhibitors. Such enzyme inhibitors include, but are not limited to, compounds such as actinonin or epiactinonin and derivatives thereof. Other enzyme inhibitors include, but are not limited to, aprotinin (Trasylol) and Bowman-Birk inhibitor.

The amount of acyclovir used in an administration composition of the present invention is an amount effective to treat the target indication. However, the amount can be less than that amount when the composition is used in a dosage unit form because the dosage unit form may contain a plurality of delivery agent compound/acyclovir, such compositions may contain a divided effective amount. The total effective amount can then be administered in cumulative units containing, in total, an effective amount of acyclovir. Moreover, those skilled in the filed will recognize that an effective amount of acyclovir will vary with many factors including the age and weight of the patient, the patient's physical condition, as well as other factors.

The total amount of acyclovir to be used of can be determined by methods known to those skilled in the art. However, because the compositions of the invention may deliver acyclovir more efficiently than compositions containing acyclovir lower amounts of acyclovir than those used in prior dosage unit forms or delivery systems can be administered to the subject, while still achieving the same blood levels and/or therapeutic effects.

According to one embodiment, the acyclovir (or a salt, ester, prodrug thereof) is administered (e.g. peripherally) at a dose of about 0.1 to about 250 mg per kilogram of body weight of the recipient per day (mg/kg/day), about 1 to about 100 mg/kg/day, or about 5 to about 20 mg/kg/day (based on the weight of acyclovir). According to another embodiment, the dose is about 10 mg/kg/day. The desired dose may be administered either as a single or divided dose.

The present invention also includes pharmaceutical compositions and dosage forms which include the aforementioned amounts of acyclovir and at least one delivery agent

Generally an effective amount of delivery agent to facilitate the delivery acyclovir is administered with acyclovir. According to one embodiment, the amount of delivery agent to acyclovir on a molar basis ranges from about 20:1 to about 1:1, from about 10:1 to about 2:1, or from about 5:1 to about 2:1.

Dosage unit forms may include any one or combination of excipients that can serve one or more functions such as: diluents, disintegrants, lubricants, plasticizers, colorants, flavorants, emulsifiers, taste-masking agents, sugars, sweeteners, salts, and dosing vehicles, including, but not limited to, water, 1,2-propane dial, ethanol, olive oil, or any combination thereof.

Disintegrants

Non-limiting examples of disintegrants include cross-linked N-vinyl-2-pyrrolidone (“CLPVP”), sodium starch glycolate, polacrilin potassium, sodium alginate, microcystalline or microfine cellulose, methyl cellulose, hydroxypropylcellulose, carboxymethyl cellulose sodium, and croscarmellose sodium. In one embodiment the disintegrant is croscarmellose sodium.

Wetting Agents

Non-limiting examples of wetting agents include alcohols, polyols, hydroxylated fatty acid esters (i.e., fatty acid esters having one or more hydroxy groups), and non-hydroxylated fatty acid esters (i.e., fatty acid esters that do not have hydroxy groups). Non-limiting examples of suitable solvents include ethanol, ethylene glycol, propylene glycol, glycerol, pentaerythritol, sorbitol, mannitol, transcutol, dimethyl isosorbide, polypropylene glycol, polyvinylalcohol, hydroxypropyl methylcellulose and other cellulose derivatives, cyclodextrins and cyclodextrin derivatives, PEG-40 hydrogenated castor oil (available as Cremophor® RH 40 from BASF Ag of Ludwigshafen, Germany), medium chain (C₈-C₁₀ fatty acids) triglycerides (available as Labrafac® CC from Gattefossé Corporation of Paramus, N.J.), oleoyl macrogol-6 glycerides (Labrafil® M 1944 CS available from Gattefossé Corporation), linoleoyl macrogol-6 glycerides (Labrafil® M 2125 CS available from Gattefossé Corporation), propylene glycol monolaurate (Lauroglycol® 90 available from Gattefossé Corporation), caprylic/capric glycerides (Imwitor® 742 available from Sasol Germany GmbH of Witten, Germany), glyceryl cocoate (Imwitor® 928 available from Sasol Germany GmbH), glyceryl caprylate (Imwitor® 988 available from Sasol Germany GmbH), propylene glycol dicaprylate/dicaprate (Miglyol® 840 from Condea Vista Co. of Cranford, N.J.), glyceryl ricinoleate (Softigen® 701 available from Sasol Germany GmbH), PEG-6 caprylic/capric glycerides (Softigen® 767 available from Sasol Germany GmbH), bis-diglycerylpolyacyladipate (Softigen® 645 available from Sasol Germany GmbH), PEG-25 trioleate (Tagat® TO available from Goldschmidt Chemical Corp, Hopewell, Va.), polysorbate 80 (Tween 80), ethers of polyethylene glycols having an average molecular weight of about 200 to about 6000 (such as tetrahydrofurfuryl alcohol PEG ether (glycofurol (BASF of Ludwigshafen, Germany, under the trademark Tetraglycol™)) or methoxy PEG (Union Carbide of Midland, Mich.)), amides and other nitrogen-containing compounds (such as 2-pyrrolidone, 2-piperidone, 1-caprolactam, N-alkylpyrrolidone, N-hydroxyalkyl-pyrrolidone, N-alkylpiperidone, N-alkylcaprolactam, dimethylacetamide and polyvinyl-pyrrolidone), esters (such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, Σ-caprolactone and isomers thereof, Δ-valerolactone and isomers thereof, and β-butyrolactone and isomers thereof), other solubilizers known in the art (such as dimethyl acetamide, dimethyl isosorbide, N-methylpyrrolidones, monooctanoin, and diethylene glycol monoethyl ether), and mixtures thereof. Other suitable wetting agents are referred to as solubilizers in U.S. Pat. No. 6,458,383, which is hereby incorporated by reference.

In one embodiment, wetting agents include, but are not limited to, polyethylene glycol having a molecular weight less than about 800 daltons (e.g., polyethylene glycol-300), propylene glycol monocaprylate (such as Capmul® PG8 (fatty acid distribution by GLC: <1.0% C₆, >98.0% C₈, <2.0% C₁₀, and <1.0% C₁₂ and higher) from Abitec Corporation of Columbus, Ohio; and Capryol 90 (containing 90% monoesters) from Gattefossé Corp., Paramus, N.J.), and mixtures thereof.

In another embodiment, wetting agents include, but are not limited to, polyethylene glycol, sodium lauryl sulfate, mixtures of glycerol and PEG (e.g. PEG-1500) esters of long-fatty acids (e.g. Gelucire® dispersion additives, such as Gelucire® 44/14 and Gelucire® 50/13, available from Gattesfosse Corp., Paramus, N.J.), mixtures of monoglycerides and diglycerides of capyrilic and capric acid in glycerol (e.g. Capmul® excipients, such as Capmul® PG8), polypropylene glycol monocaprylate (e.g. Capryol PGMC available from Gattesfosse Corp., Paramus, N.J.), soyabean oil, propylene glycol mono caprylate, caprylocaproyl polyoxylglycerides, and polysorbate 80.

Binders

Non-limiting examples of binders include methyl cellulose, microcrystalline or microfine cellulose, hydroxypropylcellulose, hydroxypropyl methylcellulose, carboxymethylcellulose, povidone, polyvinyl alcohol, gelatin, cassien, glycerine, sorbitol, mannitol, sucrose, lactose, fructose, starch, corn starch, pregelatinized starch, amylose, dextrose, amylodextrin, maltodextrin, cyclodextin, and gums (e.g. guar gum, acacia, locust bean gum, pectin, detarium micrcarpium gum, macrogol stearate, sodium alginate, and tragacanth). In one embodiment, the binder is selected from povidone, gelatin, corn starch, and pregelatinized starch.

Lubricants

Non-limiting examples of lubricants include long-chain fatty acids (e.g. stearic acid) and salts thereof (e.g. sodium stearate, potassium stearate, calcium stearate, magnesium stearate, and zinc stearate), sodium lauryl sulfate, sodium stearyl fumarate, polyethylene glycol, glyceryl behenate, fumed silica, and talc. In one embodiment the lubricant is selected from magnesium stearate, sodium lauryl sulfate, polyethylene glycol, and fumed silica.

Release Modifying Agent

Examples of release modifying agents include, but are not limited to, binders and emulsifiers such as polysorbate, sorbitan esters, medium-chain triglycerides, lecithin, mixtures of monoglycerides and diglycerides of capyrilic and capric acid in glycerol (e.g. Capmul® excipients, such as Capmul® PG8), polypropylene glycol monocaprylate (e.g. Capryol PGMC available from Gattesfosse Corp., Paramus, N.J.), diethanolamine, glyceryl monostearate, polyoxyethylene ethers, polyoxyethylene stearates, cetyl or stearyl alcohol, propylene glycol aginate, and emulsifying wax. In one embodiment the release modifying agent is selected from polysorbate, lecithin, and mixtures of monoglycerides and diglycerides of capyrilic and capric acid in glycerol.

The above-mentioned disintegrants, wetting agents, binders, lubricants and release modifying agents may serve more than one function. For example, a release modifying agent, such as lecithin, or mixtures of monoglycerides and diglycerides of capyrilic and capric acid in glycerol may also serve a wetting agent.

### Methods of Treatment

The composition of the present invention can treat any disorder which is treatable with acyclovir or its salts (e.g., acyclovir sodium) or prodrugs (e.g., valacyclovir), including those described in the Physicians' Desk Reference (58^{th} Ed., 2004, Medical Economics Company, Inc., Montvale, N.J.). Such disorders include, but are not limited to, those described above or in the patents or other publications above. Non-limiting examples are:

(1) herpes simplex 1 virus (HSV 1),

(2) herpes simplex 2 virus (HSV 2),

(3) varicella zoster virus (VZV),

(4) cytomegalovirus (CMV),

(5) Epstein-Barr virus (EBV),

(6) other herpes virus infections (e.g. feline herpes virus infections),

(7) herpetic karatitis,

(8) herpetic encaphalitis,

(9) cold sores and genital infections (caused by herpes simplex),

(10) chicken pox,

(11) shingles (caused by varicella zoster),

(12) CMV-pneumonia and retinitis, particularly in immunocompromised patients including renal and bone marrow transplant patients and patients with Acquired Immune Deficiency Syndrome (AIDS),

(13) Epstein-Barr virus (EVB) caused infectious mononucleosis, nasopharyngeal cancer, immunoblastic lymphoma, Burkitt's lymphoma and hairy leukoplakia,

(14) herpes zoster, and

(15) initial episodes and/or the management of recurrent episodes of genital herpes.

One embodiment of the present invention provides a method for administering acyclovir or a salt, ester, or prodrug thereof to an animal (preferably a mammal and more preferably a human) in need thereof, by administering the composition or dosage unit form(s) of the present invention to the animal. The preferred route of administration is oral.

Yet another embodiment is a method of treating conditions or disorders caused by a virus in an animal (preferably a mammal and more preferably a human) in need thereof by administering an effective amount of the composition or dosage unit form(s) of the present invention to the animal. In other words, an effective amount of the delivery agent compound to facilitate the delivery of the acyclovir or a salt, ester, or prodrug thereof and an effective amount (e.g., a therapeutically effective amount) of acyclovir is administered.

Yet another embodiment is a method for treating conditions or disorders caused by a virus in an animal (preferably a mammal and more preferably a human) by administering to the animal a therapeutically effective amount of the composition or dosage unit form(s) of the present invention. Such conditions and disorders, include but are not limited to, those caused by viruses of the herpes family, for example, herpes simplex 1 and 2 viruses (HSV 1 and HSV 2), varicella zoster virus (VZV), cytomegalovirus (CMV), Epstein-Barr virus (EBV), and other herpes virus infections (e.g. feline herpes virus infections).

Another embodiment is a method of treating virus infections, especially herpes infections such as herpes simplex 1 and 2 viruses (HSV 1, HSV 2), varicella zoster virus (VZV), cytomegalovirus (CMV) and Epstein-Barr virus (EBV), and other herpes virus infections (e.g. feline herpes virus infections) in a human or non-human animal by administering an effective amount of the composition or dosage unit form of the present invention.

Yet another embodiment is a method of treating clinical conditions or symptoms which are caused by the viruses enumerated above, including herpetic keratitis, herpetic encephalitis, cold sores and genital infections (caused by herpes simplex), chicken pox and shingles (caused by varicella zoster) and CMV-pneumonia and retinitis, particularly in immunocompromised patients including renal and bone marrow transplant patients and patients with Acquired Immune Deficiency Syndrome (AIDS) by administering an effective amount of the composition or dosage unit form of the present invention. Epstein-Barr virus (EVB) causes infectious mononucleosis, and is also suggested as the causative agent of nasopharyngeal cancer, immunoblastic lymphoma, Burkitt's lymphoma and hairy leukoplakia.

Yet another embodiment is a method of treating viral infections in an animal (preferably a mammal and more preferably a human) in need thereof by administering to the animal a therapeutically effective amount of the composition or dosage unit form(s) of the present invention. Generally, the viral infections are those treatable with acyclovir or a salt, ester, or prodrug thereof.

Yet another embodiment is a method for acute treatment of herpes zoster (also known as shingles) in a human in need thereof by administering (preferably orally) an effective amount of the pharmaceutical composition of the present invention. Preferably, the pharmaceutical composition is orally administered every 5 or more hours and less than 5 times daily. Preferably, the pharmaceutical composition provides bioavailability (i.e., AUC) substantially equivalent to the current acyclovir formulations marketed as ZOVIRAX® (U.S. FDA NDA No. 18828, 19909, or 20089) when 200 mg of acyclovir is administered every 4 hours 5 times daily. The treatment may be continued for 7 to 10 days.

Yet another embodiment is a method for treatment of initial episodes and/or the management of recurrent episodes of genital herpes in a human in need thereof by administering (preferably orally) an effective amount of the pharmaceutical composition of the present invention. Preferably for the treatment of initial genetic herpes, the pharmaceutical composition (e.g., 400 mg of acyclovir or a molar equivalent of a salt or prodrug thereof) is administered every 5 or more hours and less than 5 times daily. The treatment may be continued for 10 days. Preferably, the pharmaceutical composition provides bioavailability (i.e., AUC) substantially equivalent to the current acyclovir formulations marketed as ZOVIRAX® (U.S. FDA NDA No. 18828, 19909, or 20089) when 800 mg of acyclovir is administered every 4 hours 5 times daily.

Preferably for chronic suppressive therapy for recurrent genital herpes, the composition is administered once daily or less frequently. The treatment may be continued for up to 12 months, followed by re-evaluation. Preferably, the composition provides bioavailability (i.e., AUC) substantially equivalent to the current acyclovir formulations marketed as ZOVIRAX® (U.S. FDA NDA No. 18828, 19909, or 20089) when:

(1) 400 mg of acyclovir is administered 2 times daily,

(2) 200 mg of acyclovir is administered 3 times daily,

(3) 200 mg of acyclovir is administered 4 times daily, or

(4) 200 mg of acyclovir is administered 5 times daily.

Treatment may be continued for up to 12 months, followed by re-evaluation.

Yet another embodiment is a method for treatment of chickenpox in a human in need thereof by administering (preferably orally) an effective amount of the composition of the present invention. Preferably the composition (e.g., 80 mg/kg/day of acyclovir or a molar equivalent of a salt or prodrug thereof) is administered every 5 or more hours and less than 4 times daily. For children (2 years of age and older), an amount of the composition can be orally administered to provide the equivalent bioavailability as 20 mg/kg per dose 4 times daily of the current acyclovir formulations marketed as ZOVIRAX® (U.S. FDA NDA No. 18828, 19909, or 20089). For adults or children over 40 kg, an amount of the composition can be orally administered to provide the equivalent bioavailability as 800 mg of the current acyclovir formulations marketed as ZOVIRAX® (U.S. FDA NDA No. 18828, 19909, or 20089) administered 4 times daily.

### EXAMPLES

The following examples illustrate the invention without limitation. All parts are given by weight unless otherwise indicated.

The following is a brief description of the methods used to form the tablets and capsules, and the dosing protocol. Specific modifications to the process are discussed in the Examples

### Methods of Preparation:

### Capsules:

Acyclovir and SNAC were separately screened through a sieve of pore size of 500 μm (USP standard sieve # 35). Afterwards, predetermined amounts of acyclovir and SNAC were blended. Other components of the formulations are added to make the final formulations using either of three described methods: (i) excipients as powders (such as, for example, sodium lauryl sulfate, or lecithin) were added to the blend of acyclovir and SNAC to make a homogenous final blend that was filled into capsules for dosing in beagles (ii) excipients in liquid or semi-solid form such as, for example, PEG 300, soyabean oil, Capmul®, Tween 80® were used without melting, in which case acyclovir/SNAC powder blend are added to obtain a final formulation in form of a paste or semi-solid, (iii) semi-solid excipients (such as, for example, Gelucire 44/14 and Gelucire 50/13) were melted at suitable temperature such as 40-50° C. and acyclovir/SNAC powder blends were added to the melted excipients. The final formulation was filed into capsules for dosing.

In all the cases, depending on the weight of the formulation per dose, formulations were filed into suitable sized hard gelatin capsules (examples capsule size 0 or 00) Alternatively, formulations can be prepared by encapsulating ingredients in soft gelatin capsules

### Tablets:

Tablet formulations were manufactured using a wet granulation process. The process involved preparation of acyclovir and SNAC powder blend and the addition of functional pregranular excipients such as lecithin, gelatin, croscarmellose sodium, or povidone. The homogenous powder blend was then granulated in a mortar and a pestle or in a high shear granulator (depending on the batch size). In most of the formulations, water was used as the granulating liquid. Examples of other granulating agents that have been used include, for example: Tween 80® solution, starch paste. The wet granules that were dried in a vacuum oven at 50° C.-55° C. until the moisture content is less than 5%. Dry granules were then milled and screened through a #35 screen to obtain granules of uniform sizes. In all the cases, dry granules were analyzed based on the moisture content and assay of acyclovir and SNAC. Extragranular excipinets were added such as fillers, disintegrants, glidants, and lubricants. Specific examples of extragranular excipients include: pregelatinized starch, croscarmellose sodium magnesium strearate and colloidal silica. After the addition of all excipients, dry granules were compressed into tablets of predetermined weight using a suitable tablet press.

Examples of various physical tests that are conducted in each formulation are: tablet hardness, tablet friability, disintegration and assay.

As an alternate to the high shear granulation process, a fluid bed granulation or a dry granulation process can be used to manufacture tablets.

### In-Vivo Studies:

Except as noted in Example 30, the pharmacokinetic profiles of all the formulations were carried out in a beagle model. Each beagle was administered acyclovir tablets or capsules, by oral gavage, formulated with SNAC and other ingredients as disclosed herein, or existing commercially available acyclovir or valacyclovir tablets for control studies. Beagles were fasted at least 8 hrs prior to dosing and were fed immediately after study was completed (i.e., all blood was already drawn). Blood samples of about 0.5 ml volume was be withdrawn from the jugular vein, before and after dosing. The time points for blood withdrawal were: −15, +5, 10, 20, 30, 40 min, 1, 1.5, 2, 3, 4, 6 hr. The blood samples were put on ice immediately after collection and then centrifuged for 10 minutes at 3000 RPM at approximately 4° C. (within 45 minutes of collection). The plasma samples were stored at a −20° C. freezer until time of analysis of acyclovir levels. Plasma acyclovir levels were analyzed by LC-MS (Liquid Chromatography Mass Spectrometry) method. The results were presented as individual acyclovir levels per beagle or as the mean (+/−SE) from a group of four beagles.

### Example 1

### Oral (Tablet) Acyclovir Formulation with SNAC

The following describes formulations with increased oral bioavailability of acyclovir. The following formulations, as oral dosage forms (tablets or capsules), were administered to beagles. Several formulations were compared to an acyclovir tablet formulation which was prepared by the process of wet granulation and comprises: 240 mg of acyclovir and 240 mg of SNAC. Table 1 shows the list of ingredients in this acyclovir tablet formulation. The SNAC used was retained on sieve number 100 (size 150 μm), which indicated that the un-micronized SNAC had dimensions that were higher than 150 μm.

Tablets were prepared by the process of wet granulation. All ingredients were screened through a sieve of pore size of 500 μm (USP standard sieve #35). The predetermined weight of each component per tablet of the formulation (table 1) was adjusted based on the batch size (number of tablets to be prepared). For instance, the amounts of acyclovir and SNAC needed to prepare 20 tablets are acyclovir 4800 mg and SNAC 4800 mg. Similarly, the weights of all other excipients were adjusted based on the batch size.

The required amounts of acyclovir and SNAC were weighed and blended for 3 minutes using a mortar and a pestle. To the blend, the required amounts of Povidone and Croscarmellose sodium were added. The powder mixture was blended using a mortar and a pestle for 5 minutes. Purified water (2 g) was added to the powder blend drop wise while mixing the powder blend to obtain wet granules. Wet granules were dried in a vacuum oven (Isotemp Model 282A; Fisher Scientific) at 55° C. for about 8 hours until the moisture level in the granules was less than 5%. The moisture level in the dry granules was measured by solid drop to a Brinkmann 737 Karl Fisher Coulometer. Dry granules were screened through a sieve of pore size 500 μm. Extragranular excipients as shown in table 1: Pregelatinized starch, Croscarmellose sodium and Magnesium stearate were added and blended for 5 minutes. The weight per dose of the final blend of (dry granules and extragranular excipients) was compressed to tablets using a caplet-shaped tool on a single punch tablet press Korsch XL 100. The targeted tablet properties are: hardness of 8-10 KP and disintegration of 6-8 minutes in water at 37° C.

TABLE 1
These excipients are incorporated into acyclovir tablet formulations
either before the process of wet granulation (intragranular)
or before tablet compression (extragranular), as noted.

| Ingredient | mg/tablet |
|---|---|
| Acyclovir (intragranular) | 240 |
| SNAC (intragranular) | 240 |
| povidone (intragranular) | 3 |
| croscarmellose sodium (intragranular) | 12 |
| pregelatinized starch (extragranular) | 93 |
| Croscarmellose sodium (extragranular) | 4 |
| magnesium stearate (extragranular) | 3 |
| Total Weight | 595 |

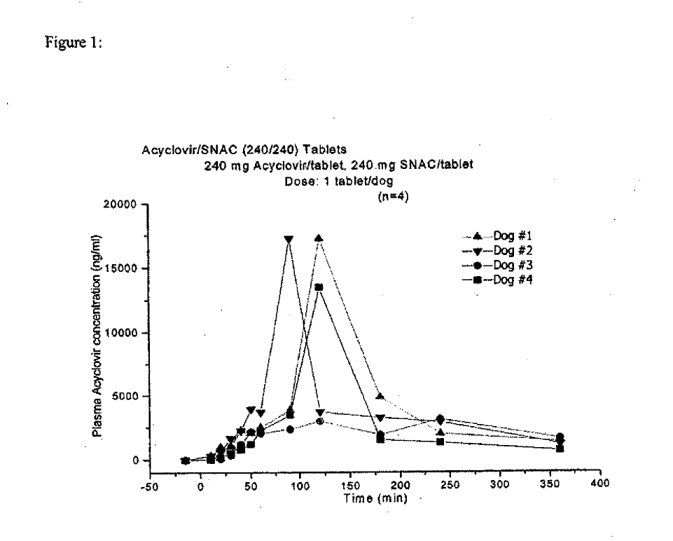

FIG. 1 shows plasma acyclovir concentrations after dosing acyclovir tablets with the components shown in Table 1 to four beagles. This data is also shown below.

| Time (min.) | Four (4) Beagles were Dosed (ng/ml) | | | |
|---|---|---|---|---|
| −15 | 0 | 0 | 0 | 0 |
| 10 | 326.6 | 328.1 | 37.5 | 40.1 |
| 20 | 996.6 | 614.4 | 75.4 | 182 |
| 30 | 1059.9 | 1655.9 | 355.1 | 547 |
| 40 | 2312.4 | 2270.8 | 1220.1 | 802.7 |
| 50 | 2076.5 | 3964.7 | 2175.4 | 1228.3 |
| 60 | 2529.6 | 3725.2 | 2071.3 | 2279.7 |
| 90 | 3891.1 | 17293 | 2389.1 | 3524.2 |
| 120 | 17293 | 3758.8 | 3015.3 | 13531.8 |
| 180 | 4886.3 | 3289.2 | 1951.9 | 1537.3 |
| 240 | 2033.3 | 2934.3 | 3164.7 | 1330 |
| 360 | 1414 | 1221.2 | 1643 | 696.7 |

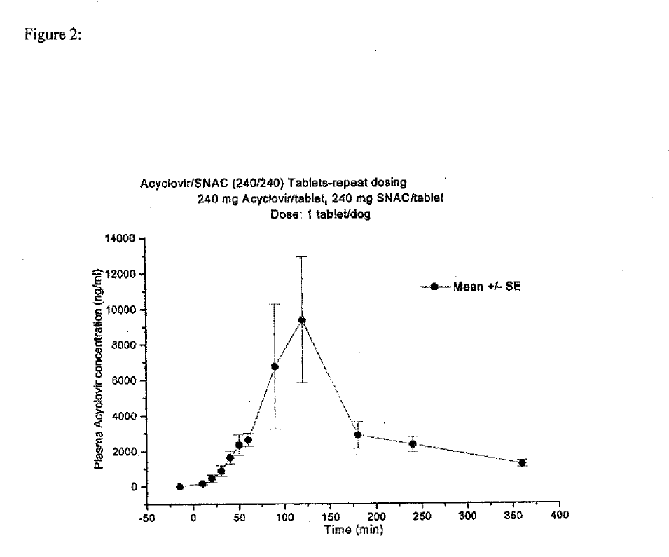

FIG. 2 shows mean plasma acyclovir levels after dosing acyclovir capsules formulated the compounds of Table 1. Each data point represents the mean+/−SE (n=4 beagles).

### Example 2

### Oral (Tablet) Acyclovir Formulation with Micronized SNAC

The formulation is the same as that in Example 1, except that the SNAC was micronized before being combined with acyclovir. The un-micronized SNAC of Example 1 was retained on sieve number 100 (size 150 μm) which indicated that the un-micronized SNAC has dimensions that are higher than 150 μm. The particle size of the delivery agent compound (I.e. SNAC) in this example was reduced by using Ball Mill (Retsch Ball Mill, manufactured by GlenMills, Clifton, N.J.) and subsequently by sieving through sieve number 140 (sieve size 106 μm). Accordingly the size of the micronized SNAC was less than 106 μm. Additional analysis by scanning electron micrograph (SEM), indicated that prior to size reduction, SNAC has a cylindrical-shaped morphology that was converted to spherical-morphology by micronization.

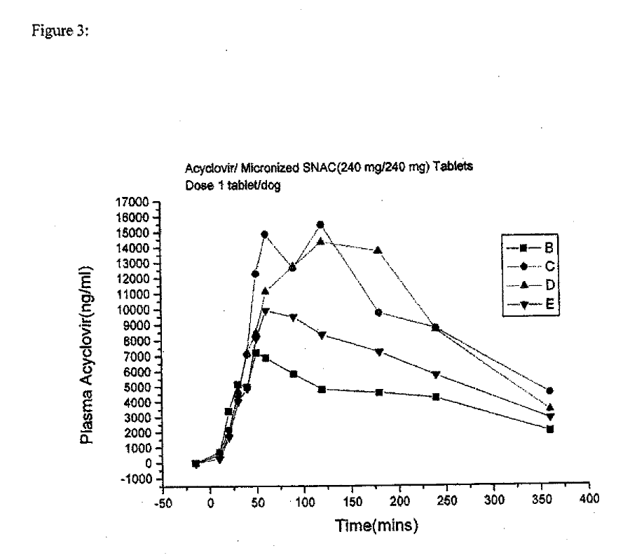

FIG. 3 shows plasma acyclovir concentrations after dosing Acyclovir tablets made with micronized SNAC. Acyclovir/SNAC (240 mg/240 mg) per tablet. One tablet was dosed per beagle. This data is also shown below:

| Time (min.) | Four (4) Beagles were Dosed (ng/ml) | | | |
|---|---|---|---|---|
| −15 | 0 | 0 | 0 | 0 |
| 10 | 512 | 702.2 | 523.5 | 270.5 |
| 20 | 3396.4 | 2178.2 | 1860.3 | 1703.1 |
| 30 | 5150.1 | 4434.1 | 4738.4 | 4045.4 |
| 40 | 4984.4 | 7136 | 7216.1 | 4891.4 |
| 50 | 7214 | 12284.8 | 8500.8 | 8137.2 |
| 60 | 6883.7 | 14860.7 | 11136.3 | 9920.7 |
| 90 | 5827.3 | 12665.8 | 12750.9 | 9519.2 |
| 120 | 4816.4 | 15474.6 | 14356.9 | 8366.1 |
| 180 | 4575 | 9750.8 | 13748.1 | 7256 |
| 240 | 4216.8 | 8767 | 8679.9 | 5734.5 |
| 360 | 1971.7 | 4536.6 | 3411.2 | 2856.3 |

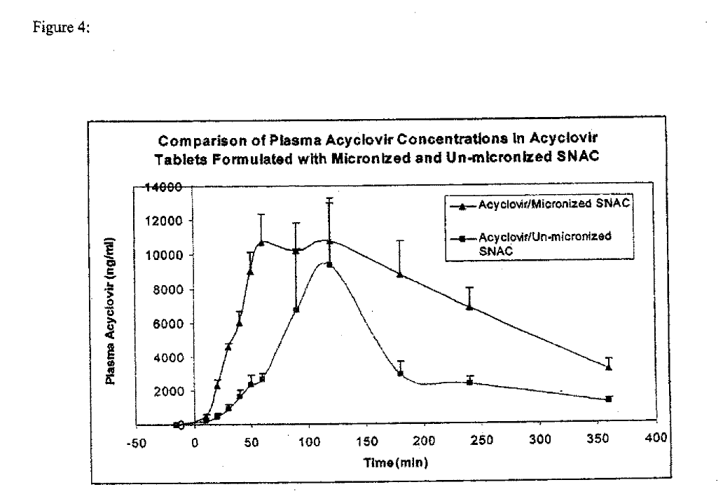

FIG. 4 shows the mean plasma acyclovir levels after dosing acyclovir tablets formulated with micronized SNAC (Example 2) and un-micronized SNAC (Example 1).

### Example 3

### Oral (Capsule) Acyclovir Formulations with a Muco-Adhesive Agent (Carbopol 934P)

The sodium salt of Carbopol 934P (CP) (prop-2-enoic acid) having an average molecular weight of about 300,000 and a high viscosity at low concentrations was used in this Example. This was prepared by freeze-drying a 0.5% solution of Carbopol that was neutralized by 10M sodium hydroxide solution to pH 7.0. The predetermined weights (tables 2 and 3) of acyclovir and SNAC were blended for 3 minutes using a mortar and a pestle. The required weight of Carbopol 934P was added and the powder mixture was blended for 3 minutes. The final powder blend was sized into a hard gelatin capsule of size 00 at the weight/dose as shown in tables 2 and 3. Capsules were administered to beagles.

TABLE 2
Components of Acyclovir/SNAC formulation according to Example 3

| Ingredient | mg/capsule |
|---|---|
| Acyclovir | 240 |
| SNAC | 240 |
| Carbopol 934P | 0.96 |
| Total Weight | 480.96 |

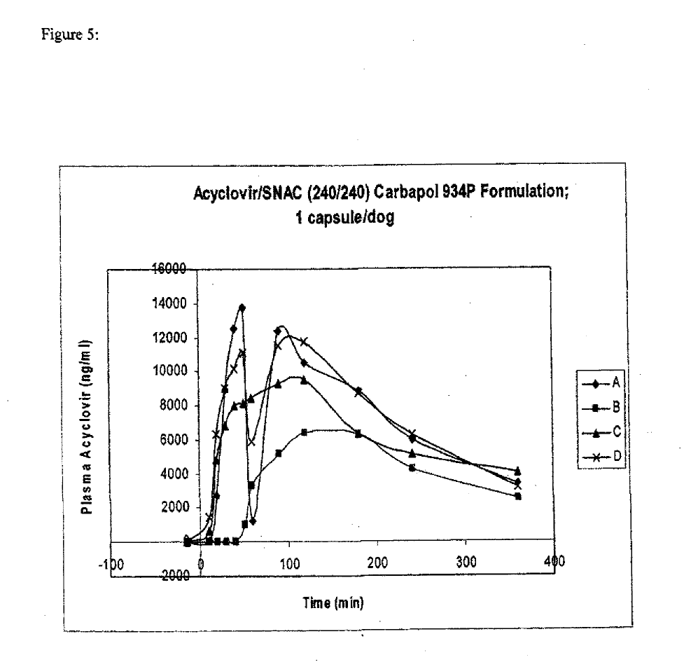

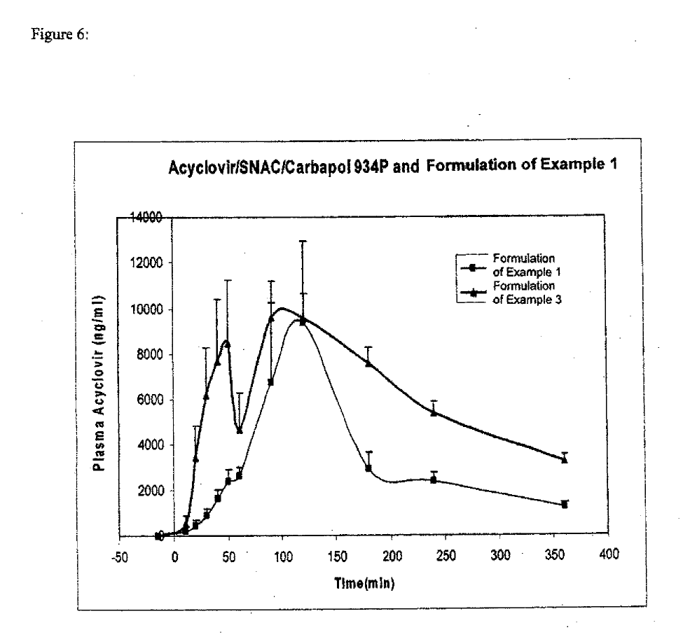

FIG. 5 shows plasma acyclovir Concentrations after dosing acyclovir/SNAC made with Carbopol 934P in beagles (beagles: A, B, C, D).
FIG. 6 shows a comparison of the acyclovir-Carbopol formulation to the formulation in Example 1.

TABLE 3
Components of acyclovir formulation made with 0.8 wt %
Carbopol 934P (sodium salt).

| Ingredients | mg/capsule | |
|---|---|---|
| Acyclovir | 240 | |
| SNAC | 240 | |
| Carbopol 934P | 4.0 | |
| Total Weight | 484 | mg |

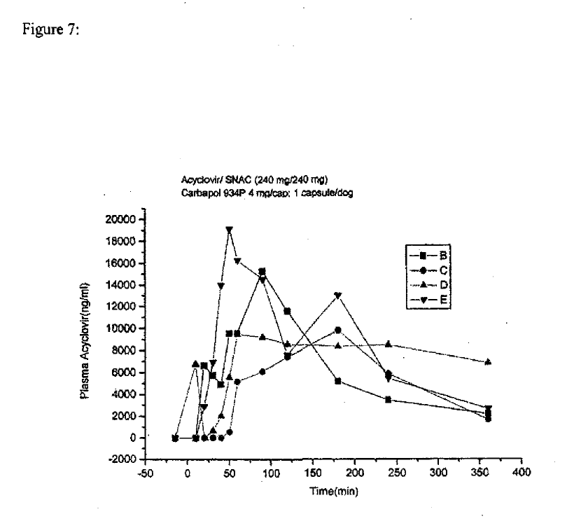

FIG. 7 shows the pharmacokinetic profiles of acyclovir capsules formulated with 0.8% Carbopol 934P® when administered to 4 beagles. The data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 0 | 0 | 6785.5 | 0 |
| 20 | 6657.7 | 0 | 0 | 2892.9 |
| 30 | 5760.5 | 0 | 685.2 | 6958.4 |
| 40 | 4937.4 | 0 | 2028.1 | 14003.8 |
| 50 | 9564.5 | 550.8 | 5580.2 | 19174.5 |
| 60 | 9566.1 | 5185.7 | 9497.9 | 16292.7 |
| 90 | 15287.8 | 6103.8 | 9228.7 | 14549.8 |
| 120 | 11598 | 7427.9 | 8600 | 7627.9 |
| 180 | 5188.3 | 9836.4 | 8382 | 13025.6 |
| 240 | 3454.9 | 5886.2 | 8516.7 | 5463.9 |
| 360 | 2176.4 | 1654.5 | 6868.9 | 2665.3 |

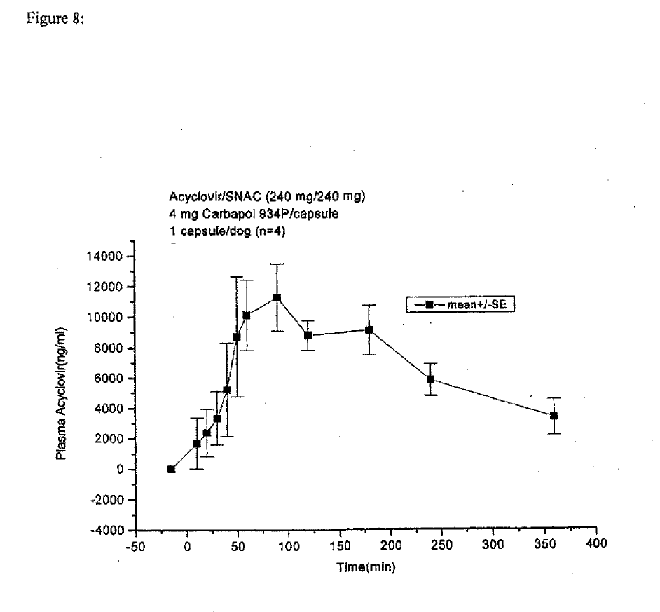

FIG. 8 shows mean plasma acyclovir levels after dosing acyclovir capsules formulated with 0.8% Carbopol 934P. Each data point represents the mean+/−SE (n=4 beagles).

While it is not intended that the invention be limited or bound to any particular theory, muco-adhesive agents, such as Carbopol 934P, may improve oral bioavailability of acyclovir due to prolonged residence time in the gastrointestinal tract due to adherence to the gastrointestinal mucosa.

### Example 4

### Oral (Tablet) Acyclovir Formulation with Low Molecular Weight Gelatin

Tablets were prepared by the process of wet granulation. Micronized SNAC was used in the formulation that was obtained by size reduction by Ball Mill (Retsch Ball Mill—GlenMills, Clifton, N.J.) and subsequently by sieving through sieve number 140 (sieve size 106 μm). All other ingredients were screened through a sieve of pore size of 500 μm (USP standard sieve #35). The predetermined weight of each component per tablet of the formulation (table 4) was adjusted based on the batch size (number of tablets to be prepared). For instance, the amounts of acyclovir and SNAC needed to prepare 20 tablets are acyclovir 4800 mg and SNAC 4800 mg. Accordingly, the weights of all other excipients were adjusted based on the batch size.

The required amounts of acyclovir and SNAC were weighed and blended for 3 minutes using a mortar and a pestle. To the blend, the required amounts of gelatin and croscarmellose sodium were added. The powder mixture was blended using a mortar and a pestle for 5 minutes. Purified water (2 g) was added to the powder blend drop wise while mixing the powder blend to obtain wet granules. Wet granules were dried in a vacuum oven (Isotemp Model 282A; Fisher Scientific) at 55° C. for about 8 hours until the moisture level in the granules was less than 5%. The moisture level in the dry granules was measured by solid drop to a Brinkmann 737 Karl Fisher Coulometer. Dry granules were screened through a sieve of pore size 500 μm. Extragranular excipients as shown in table 4: Pregelatinized starch, Croscarmellose sodium and Magnesium stearate were added and blended for 5 minutes. The weight per dose of the final blend of (dry granules and extragranular excipients) was compressed to tablets using a caplet-shaped tool on a single punch tablet press Korsch XL100. The targeted tablet properties are: hardness of 8-10 KP and disintegration time of 6-8 minutes in water at 37° C.

TABLE 4

| | 5% Gelatin | 10% Gelatin |
|---|---|---|
| Ingredients | (mg/tablet) | (mg/tablet) |
| Acyclovir (intragranular) | 240 | 240 |
| SNAC (intragranular) | 240 | 240 |
| Gelatin (intragranular) | 24 | 48 |
| Croscarmellose sodium (intragranular) | 12 | 12 |
| Croscarmellose sodium (extragranular) | 6 | 6 |
| Pregelatinized starch (extragranular) | 75 | 75 |
| Magnesium stearate (extragranular) | 6 | 6 |
| Total Weight | 603 | 627 |

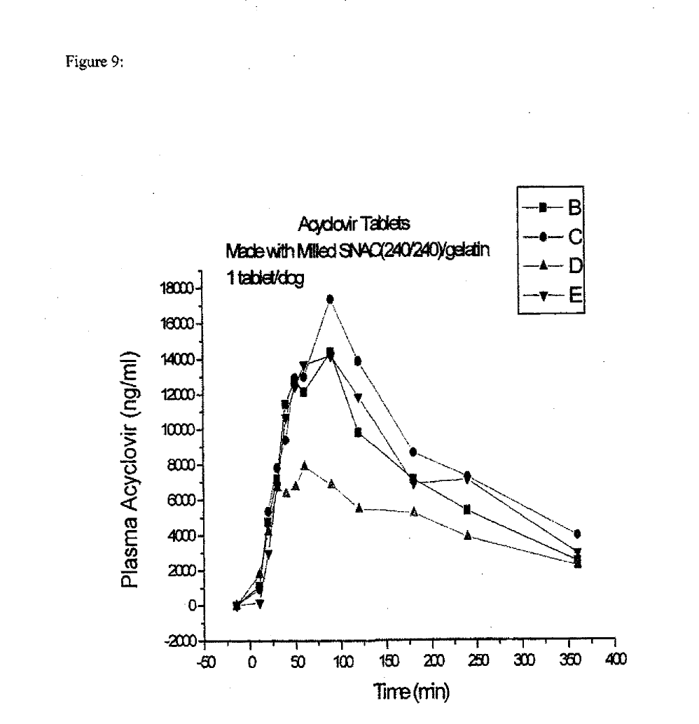

FIG. 9 shows plasma acyclovir concentrations in beagles after dosing acyclovir tablets formulated with 5% low molecular weight gelatin. Each tablet contained acyclovir 240 mg/SNAC 240 mg. One tablet was dosed per beagle. This data is also shown below.

| | |
|---|---|
| Time | |
| (min.) | Four (4) Beagles were Dosed |

| | | | | |
|---|---|---|---|---|
| −15 | 0 | 0 | 0 | 0 |
| 10 | 1098.7 | 943.1 | 1793.9 | 160.2 |
| 20 | 4754.2 | 5357.5 | 4221 | 2999 |
| 30 | 7198.6 | 7836 | 6720.6 | 6821.5 |
| 40 | 11455.9 | 9402.7 | 6372.1 | 10689.9 |
| 50 | 12772.5 | 12954.3 | 6755 | 12436.4 |
| 60 | 12137.4 | 12997.3 | 7911.2 | 13700.1 |
| 90 | 14392.3 | 17373.1 | 6847.6 | 14194.8 |
| 120 | 9824.9 | 13901.7 | 5500.5 | 11863.6 |
| 180 | 7144.9 | 8667.6 | 5244.2 | 6887.9 |
| 240 | 5358.6 | 7290.9 | 3877.5 | 7117.2 |
| 360 | 2486.1 | 3954.5 | 2228.5 | 2939.3 |

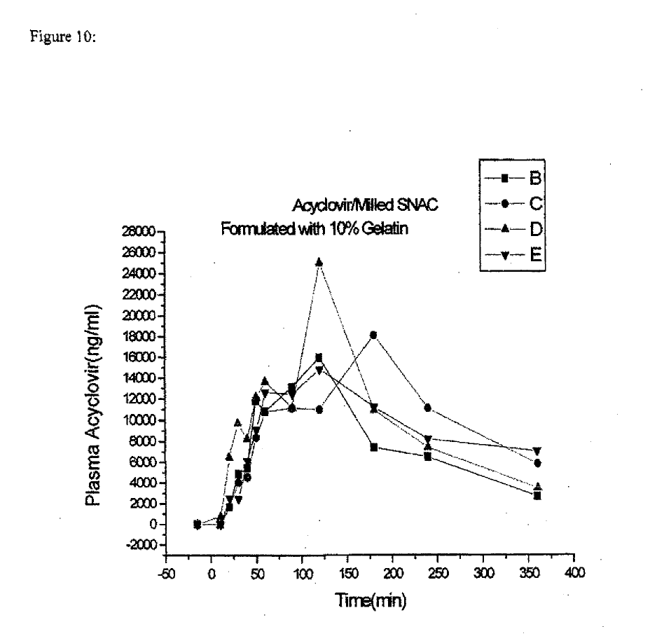

FIG. 10 shows plasma acyclovir concentrations in beagles after dosing acyclovir tablets formulated with 10% low molecular weight gelatin. Each tablet contained acyclovir 240 mg/SNAC 240 mg. One tablet was dosed per beagle. This data is also shown below.

| | |
|---|---|
| Time | |
| (min.) | Four (4) Beagles were Dosed |

| | | | | |
|---|---|---|---|---|
| −15 | 0 | 0 | 0 | 0 |
| 10 | 0 | 0 | 777.9 | 0 |
| 20 | 1659.6 | 1693.6 | 6506.6 | 2511.6 |
| 30 | 4884 | 4055.9 | 9746.1 | 2466.6 |
| 40 | 5464.1 | 4573.9 | 8288 | 6125.6 |
| 50 | 11831.2 | 8382.1 | 12175.8 | 9149.8 |
| 60 | 10831.5 | 10843.2 | 13693 | 12636.5 |
| 90 | 13155.5 | 11148.7 | 11232.8 | 12503.3 |
| 120 | 16011.6 | 11039.3 | 25098.6 | 14879.6 |
| 180 | 7405.6 | 18144.8 | 10988.2 | 11244.4 |
| 240 | 6499 | 11134.1 | 7441.4 | 8235.1 |
| 360 | 2671.9 | 5845.1 | 3514.1 | 7067.3 |

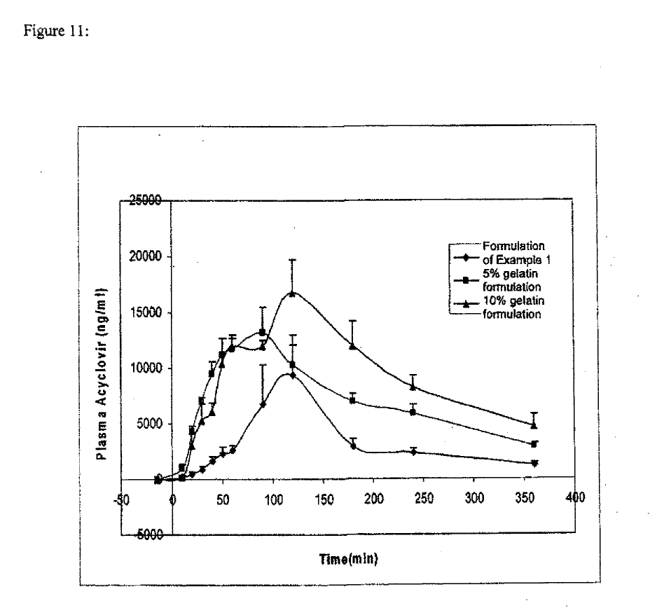

FIG. 11 shows a comparison of the pharmacokinetic profiles to the acyclovir tablet formulation in Example 1.

### Example 5

### Oral (Tablet) Acyclovir Formulation

Tablets were prepared by the process of wet granulation as described in Example 4, except that un-micronized SNAC was used in place of micronized SNAC, and povidone was used in place of gelatin. Amounts of ingredients are as shown below in Table 5.

TABLE 5

| Ingredients | mg/tablet | |
|---|---|---|
| Acyclovir | 80 | |
| SNAC | 240 | |
| Kollidon 90F (Povidone k90), | 2.0 | |
| (intragranular, 0.5%) | | |
| *Ac-Di-Sol (Croscarmellose | 8.0 | |
| sodium)(Intragranular, 2.0%) | | |
| *Ac-Di-Sol (Croscarmellose | 4.0 | |
| sodium)(Extragranular, 1.0%) | | |
| Pre-gelatinized Starch (Starch 1500 ®) | q.s | |
| Magnesium Stearate (1% w/w) | 4.0 | |
| Total | 400 | mg |

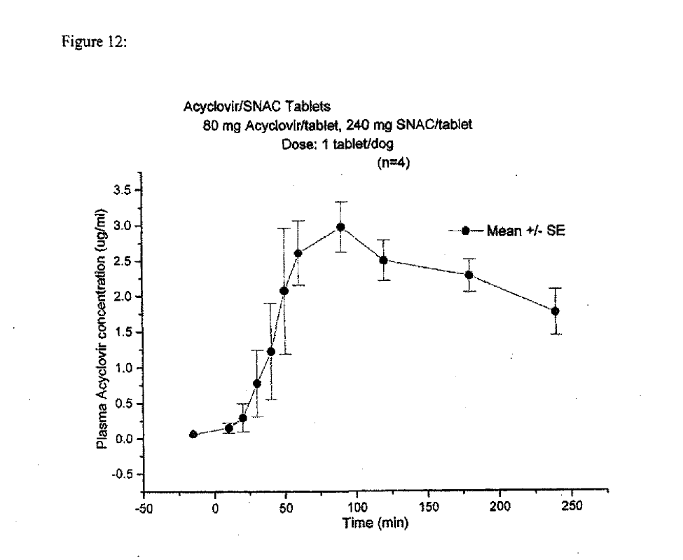

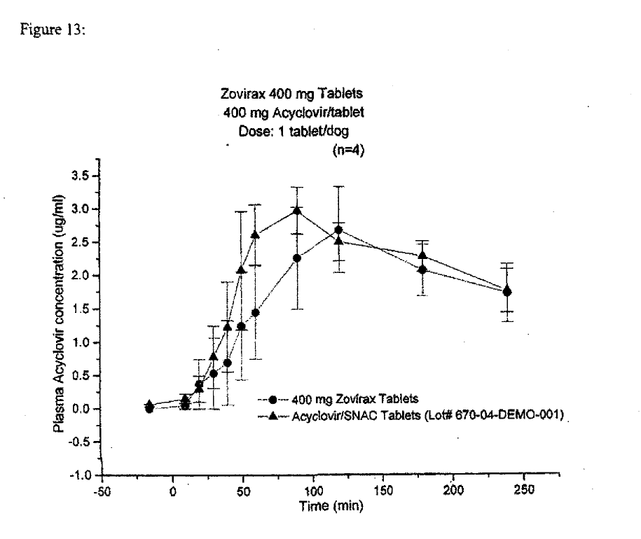

FIG. 12 shows mean plasma acyclovir levels after dosing.
FIG. 13 shows mean plasma acyclovir levels after dosing compared with 400 mg ZOVIRAX® tablets.

### Example 6

### Oral (Tablet) Acyclovir Formulation

Tablets were prepared by the process of wet granulation as described in Example 4, except that that un-micronized SNAC was used in place of micronized SNAC, povidone was used in place of gelatin, and anhydrous emcompress (dibasic calcium phosphate) was used in place of pregelatinized starch. Ingredient amounts are as shown in Table 6.

TABLE 6

| Ingredients | mg/tablet | |
|---|---|---|
| Acyclovir | 80 | |
| SNAC | 240 | |
| Kollidon 90F (Povidone k90), | 8.0 | |
| (intragranular, 2.0%) | | |
| *Ac-Di-Sol (Croscarmellose | 8.0 | |
| sodium)(Intragranular, 2.0%) | | |
| *Ac-Di-Sol (Croscarmellose | 4.0 | |
| sodium) (Extragranular, 1.0%) | | |
| Anhydrous Emcompress | q.s | |
| Magnesium Stearate (1% w/w) | 4.0 | |
| Total | 400 | mg |

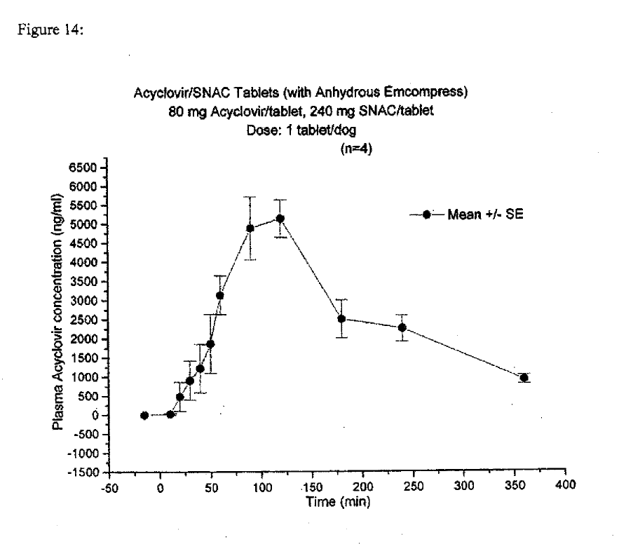

FIG. 14 shows mean plasma acyclovir levels after dosing acyclovir/SNAC tablets. Each data point represents the mean+/−SE (n=4 beagles).

### Example 7

### Oral (Tablet) Acyclovir Formulation

Tablets were prepared by the process of wet granulation as described in Example 4, except that that un-micronized SNAC was used in place of micronized SNAC, corn starch was used in place of gelatin, and anhydrous Emcompress® (dibasic calcium phosphate) was used in place of pregelatinized starch. Ingredient amounts are as shown in Table 7.

TABLE 7

| Ingredients | mg/tablet | |
|---|---|---|
| Acyclovir | 80 | |
| SNAC | 240 | |
| Corn Starch (intragranular, 1.0%) | 4.0 | |
| *Ac-Di-Sol (Croscarmellose | 8.0 | |
| sodium)(Intragranular, 2.0%) | | |
| *Ac-Di-Sol(Croscarmellose | 4.0 | |
| sodium)(Extragranular, 1.0%) | | |
| Anhydrous Emcompress ® | q.s | |
| Magnesium Stearate (1% w/w) | 4.0 | |
| Total | 400 | mg |

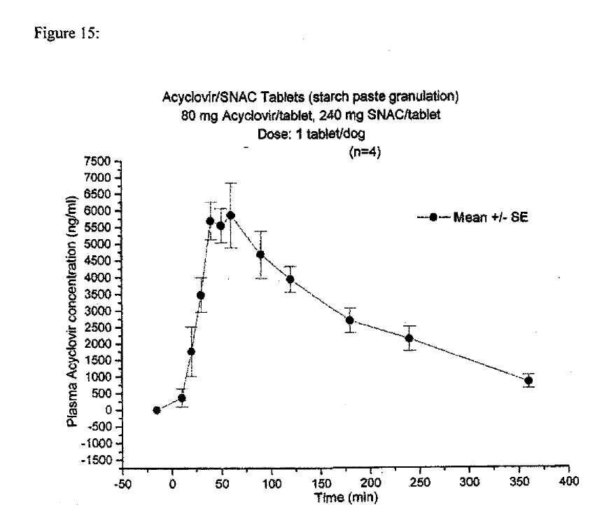

FIG. 15 shows mean plasma acyclovir levels after dosing acyclovir/SNAC tablets. Each data point represents the mean+/−SE (n=4 beagles).

### Example 8

### Oral (Tablet) Acyclovir Formulation

Tablets were prepared by the process of wet granulation as described in Example 4, except that that un-micronized SNAC was used in place of micronized SNAC, and povidone was used in place of gelatin. Ingredient amounts are as shown in Table 8.

TABLE 8

| Ingredients | mg/tablet | |
|---|---|---|
| Acyclovir | 240 | |
| SNAC | 240 | |
| Kollidon 90F(Povidone k90), | 3.0 | |
| (intragranular, 0.5%) | | |
| *Ac-Di-Sol(Croscarmellose | 12.0 | |
| sodium)(Intragranular, 2.0%) | | |
| *Ac-Di-Sol (Croscarmellose | 6.0 | |
| sodium)(Extragranular, 1.0%) | | |
| Pre-gelatinized Starch (Starch 1500 ®) | q.s | |
| Magnesium Stearate (1% w/w) | 6.0 | |
| Total | 600 | mg |

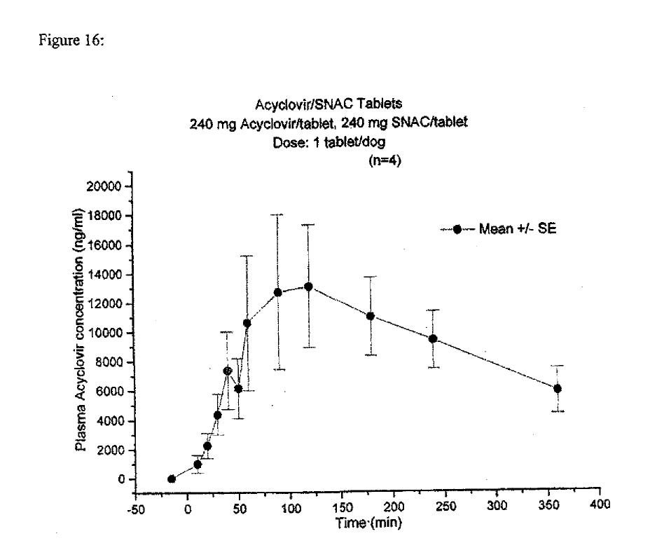

FIG. 16 shows mean plasma acyclovir levels after dosing acyclovir/SNAC tablets. Each data point represents the mean+/−SE (n=4 beagles).

### Example 9

### Oral (Tablet) Acyclovir Formulation

Tablets were prepared by the process of wet granulation as described in Example 4, except that that un-micronized SNAC was used in place of micronized SNAC, and povidone was used in place of gelatin. Ingredient amounts are as shown in Table 9.

TABLE 9

| Ingredients | mg/tablet | |
|---|---|---|
| Acyclovir | 240 | |
| SNAC | 240 | |
| Kollidon 90F(Povidone k90), | 12.0 | |
| (intragranular, 2%) | | |
| *Ac-Di-Sol(Croscarmellose | 12.0 | |
| sodium)(Intragranular, 2.0%) | | |
| *Ac-Di-Sol(Croscarmellose | 6.0 | |
| sodium)(Extragranular, 1.0%) | | |
| Anhydrous Emcompress | q.s | |
| Magnesium Stearate (1% w/w) | 6.0 | |
| Total | 600 | mg |

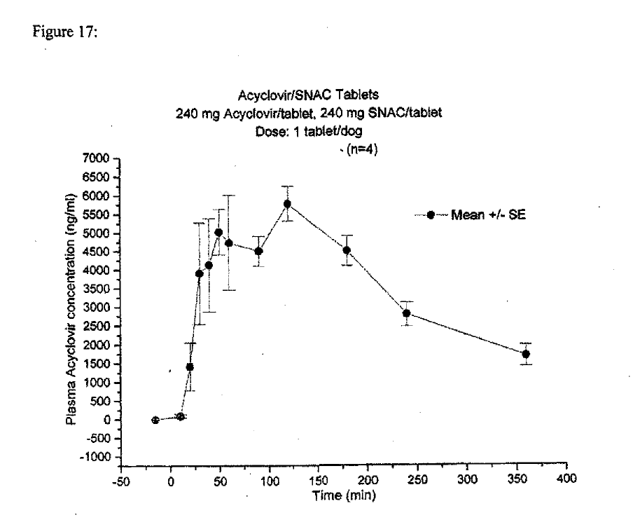

FIG. 17 shows mean plasma acyclovir levels after dosing acyclovir/SNAC tablets. Each data point represents the mean+/−SE (n=4 beagles).

### Example 10

### Oral (Tablet) Acyclovir Formulation

Tablets were prepared by the process of wet granulation as described in Example 4, except that that un-micronized SNAC was used in place of micronized SNAC, and povidone was used in place of gelatin. Ingredient amounts are as shown in Table 10.

TABLE 10

| Ingredients | mg/tablet | |
|---|---|---|
| Acyclovir | 400 | |
| SNAC | 240 | |
| Kollidon 90F(Povidone k90), | 4.0 | |
| intragranular, 0.5%) | | |
| *Ac-Di-Sol(Croscarmellose | 16.0 | |
| sodium)(Intragranular, 2.0%) | | |
| *Ac-Di-Sol(Croscarmellose | 8.0 | |
| sodium)(Extragranular, 1.0%) | | |
| Pre-gelatinized Starch (Starch 1500 ®) | q.s | |
| Magnesium Stearate (1% w/w) | 8.0 | |
| Total | 800 | mg |

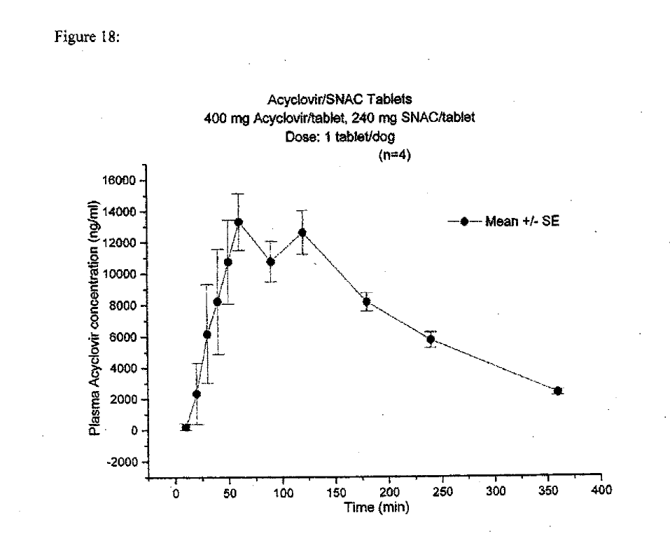

FIG. 18 shows mean plasma acyclovir levels after dosing acyclovir/SNAC tablets. Each data point represents the mean+/−SE (n=4 beagles).

### Example 11

### Oral (Tablet) Acyclovir Formulation

Tablets were prepared by the process of wet granulation as described in Example 4, except that that un-micronized SNAC was used in place of micronized SNAC, and povidone was used in place of gelatin. Ingredient amounts are as shown in Table 11.

TABLE 11

| Ingredients | mg/tablet | |
|---|---|---|
| Acyclovir | 240 | |
| SNAC | 80 | |
| Kollidon 90F(Povidone k90), | 2.0 | |
| intragranular, 0.5%) | | |
| *Ac-Di-Sol (Croscarmellose | 8.0 | |
| sodium)(Intragranular, 2.0%) | | |
| *Ac-Di-Sol(Croscarmellose | 4.0 | |
| sodium)(Extragranular, 1.0%) | | |
| Pre-gelatinized Starch (Starch 1500 ®) | q.s | |
| Magnesium Stearate (1% w/w) | 4.0 | |
| Total | 400 | mg |

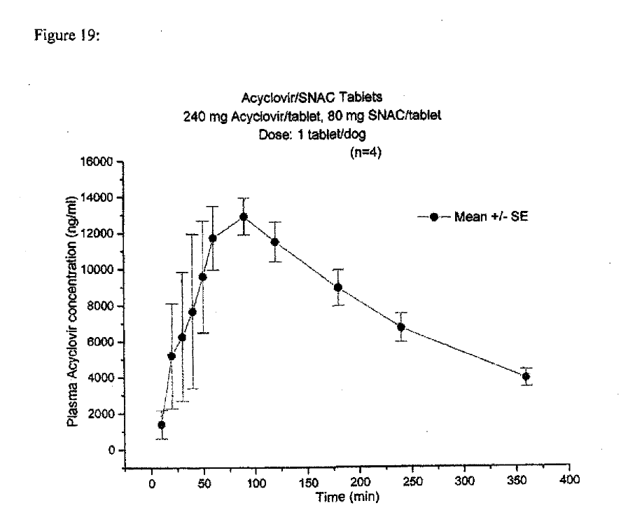

FIG. 19 shows mean plasma acyclovir levels after dosing acyclovir/SNAC tablets. Each data point represents the mean+/−SE (n=4 beagles).

### Example 12

### Oral (Tablet) Acyclovir Formulation

Tablets were prepared by the process of wet granulation as described in Example 4, except that that un-micronized SNAC was used in place of micronized SNAC, and povidone was used in place of gelatin. Also, an aqueous solution of polysorbate 80 was prepared and used as the granulating solution instead of purified water. To prepare polysorbate 80 solution, 0.5 g of polysorbate was weighed and dissolved in 7.5 g of water by gentle stirring on a magnetic stirrer. For a tablet formulation batch of 20 tablets, 1.536 g of polysorbate 80 solution will contain 96 mg of polysorbate 80 that was needed. Ingredient amounts are as shown in Table 12.

TABLE 12

| Ingredients | mg/tablet | |
|---|---|---|
| Acyclovir | 240 | |
| SNAC | 240 | |
| Polysorbate 80 | 4.8 | |
| Kollidon 90F(Povidone k90), | 3.0 | |
| intragranular) | | |
| *Ac-Di-Sol (Croscarmellose | 12 | |
| sodium)(Intragranular) | | |
| *Ac-Di-Sol(Croscarmellose | 4.99 | |
| sodium)(Extragranular) | | |
| Pre-gelatinized Starch (Starch 1500 ®) | q.s | |
| Magnesium Stearate | 4.99 | |
| Total | 580 | mg |

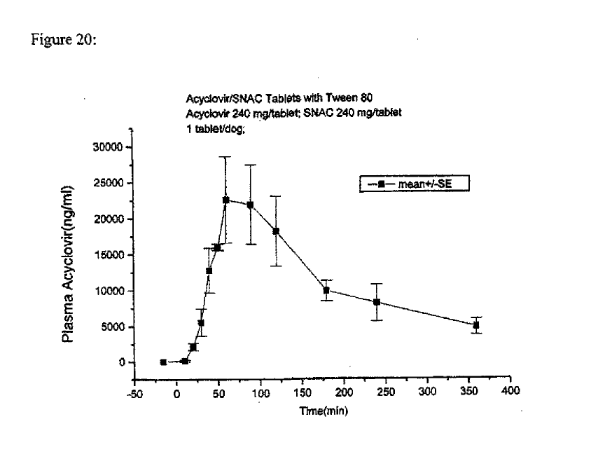

FIG. 20 shows mean plasma acyclovir levels after dosing acyclovir/SNAC tablets. Each data point represents the mean+/−SE (n=4 beagles).

### Example 13

### Oral (Tablet) Acyclovir Formulation

Tablets were prepared by the process of wet granulation as described in Example 4, except that that un-micronized SNAC was used in place of micronized SNAC, and 9.6 mg/tablet of sodium lauryl sulfate was added to the blend during the wet granulation processing step. Amounts of ingredients are as shown in Table 13.

TABLE 13

| Ingredients | mg/tablet | |
|---|---|---|
| Acyclovir | 240 | |
| SNAC | 240 | |
| Sodium lauryl sulfate | 9.6 | |
| Gelatin | 48 | |
| Ac-Di-Sol (Croscarmellose | 12 | |
| sodium)(Intragranular) | | |
| *Ac-Di-Sol (Croscarmellose | 5.5 | |
| sodium)(Extragranular) | | |
| Pre-gelatinized Starch (Starch 1500 ®) | q.s | |
| Magnesium Stearate (extragranular) | 5.5 | |
| Total | 600 | mg |

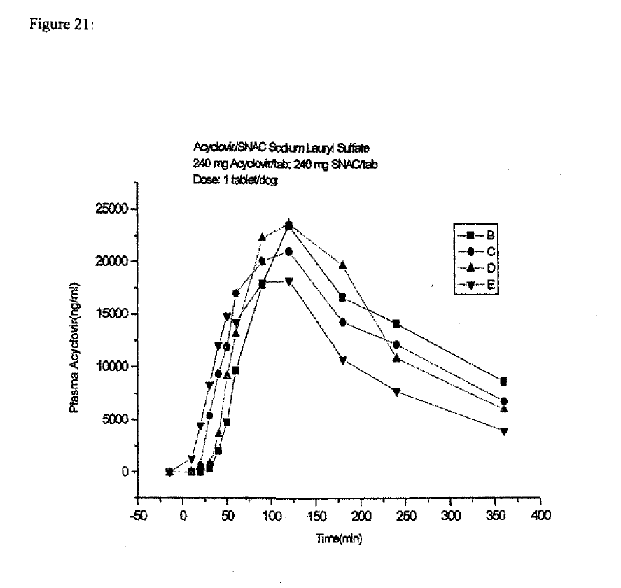

FIG. 21 shows the pharmacokinetic profiles of acyclovir tablets formulated with gelatin and sodium lauryl sulfate. This data is also shown below.

TABLE 21A

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 0 | 0 | 0 | 1324.6 |
| 20 | 0 | 608.6 | 556.6 | 4432.7 |
| 30 | 311.1 | 5362.2 | 832.5 | 8329.3 |
| 40 | 2022.8 | 9361.4 | 3636 | 12061.6 |
| 50 | 4783.2 | 11910.2 | 9160.1 | 14809.6 |
| 60 | 9652.3 | 17007.8 | 13132.4 | 14246.8 |
| 90 | 17833.3 | 20074.7 | 22294.1 | 18026.1 |
| 120 | 23518.1 | 21053.9 | 23703.7 | 18271.5 |
| 180 | 16675 | 14285.7 | 19715.4 | 10764.1 |
| 240 | 14126.5 | 12165.9 | 10854.4 | 7757.9 |
| 360 | 8650.5 | 6799.6 | 6028.5 | 3944.5 |

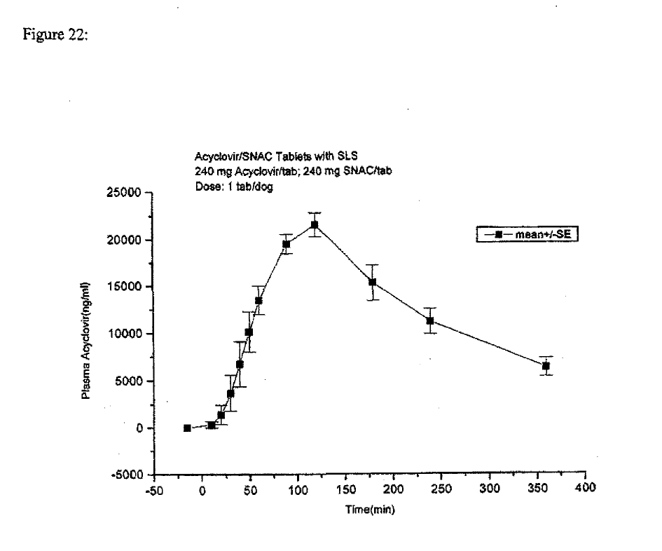

FIG. 22 shows the mean plasma acyclovir levels after dosing acyclovir tablets formulated with gelatin and sodium lauryl sulfate. Each data point represents the mean+/−SE (n=4 beagles).

### Example 14

### Oral (Tablet) Acyclovir Formulation

Tablets were prepared by the process of wet granulation as described in Example 4, except that that un-micronized SNAC was used in place of micronized SNAC, and lecithin, and povidone were added to the blend during the wet granulation processing step in place of gelatin. Amounts of ingredients are as shown in Table 14.

TABLE 14

| Ingredients | mg/dose | |
|---|---|---|
| Acyclovir | 240 | |
| SNAC | 240 | |
| Lecithin (Soybean) | 48 | |
| Kollidon 90F (povidone), (intragranular) | 3.0 | |
| *Ac-Di-Sol (Croscarmellose | 12.0 | |
| sodium) (Intragranular) | | |
| *Ac-Di-Sol (Croscarmellose | 5.43 | |
| sodium) (Extragranular) | | |
| Pre-gelatinized Starch (Starch 1500 ®) | q.s | |
| Magnesium Stearate | 5.43 | |
| Total | 620 | mg |

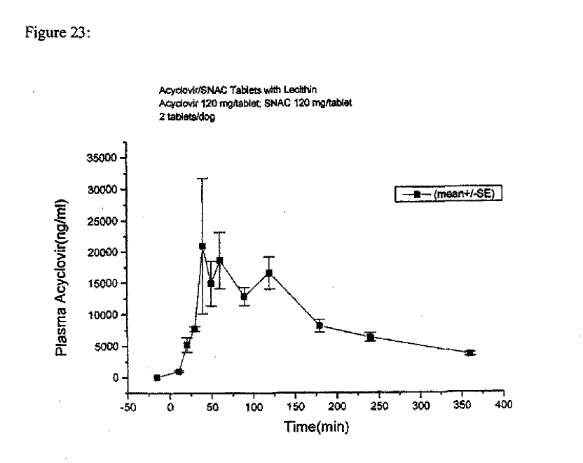

FIG. 23 shows mean plasma acyclovir levels after dosing acyclovir/SNAC tablets. Each data point represents the mean+/−SE (n=4 beagles).

### Example 15

### Oral (Capsule) Acyclovir Formulation

Acyclovir and SNAC were separately screened through a sieve of pore size of 500 μm (USP standard sieve # 35). Afterwards, predetermined amounts (table 15) of acyclovir and SNAC were mixed with gelatin in a mortar and a pestle for 3 minutes. In a beaker, Gelucire 44/14 was melted at 40° C. on a hot plate and mixed with the required amounts of soyabean oil. The mixture of Gelucire and soyabean oil was gently added to the powder blend while mixing in a mortar and pestle. The final blend was mixed for 3 minutes and later filled into a hard gelatin capsule.

TABLE 15

| Ingredients | mg/dose | |
|---|---|---|
| Acyclovir | 80 | |
| SNAC | 240 | |
| Gelatin | 28 | |
| Gelucire 44/14 | 240 | |
| Soybean Oil | q.s | |
| Total | 600 | mg |

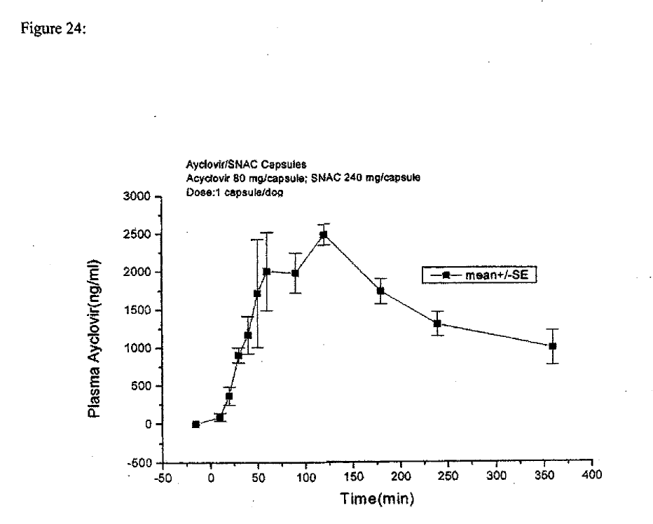

FIG. 24 shows mean plasma acyclovir levels after dosing acyclovir/SNAC tablets. Each data point represents the mean+/−SE (n=4 beagles).

### Example 16

### Oral (Capsule) Acyclovir Formulation

The method of Example 15 was repeated except that PEG 300 and Capmul PG-8 were used in place of gelatin and Gelucire 44/14 in the amounts shown below in Table 16:

TABLE 16

| Ingredients | mg/dose |
|---|---|
| Acyclovir | 240 |
| SNAC | 80 |
| PEG 300 | 291.2 |
| Capmul PG-8 | 108.9 |
| Soybean Oil | q.s |
| Total | 733.17 |

### Example 17

### Oral (Capsule) Acyclovir Formulation

The method of Example 15 was repeated except PEG 300, and Capmul PG-8 were used in place of gelatin and Gelucire 44/14 in the amounts shown below in Table 17:

TABLE 17

| Ingredients | mg/dose |
|---|---|
| Acyclovir | 240 |
| SNAC | 80 |
| PEG 300 | 108.9 |
| Capmul PG-8 | 291.2 |
| Soybean Oil | q.s |
| Total | 733.17 |

### Example 18

### Oral (Capsule) Acyclovir Formulation

The method of Example 15 was repeated except PEG 300, and Capmul PG-8 were used in place of gelatin and Gelucire 44/14 in the amounts shown below in Table 18:

TABLE 18

| Ingredients | mg/dose |
|---|---|
| Acyclovir | 240 |
| SNAC | 80 |
| PEG 300 | 203.16 |
| Capmul PG-8 | 542.44 |
| Soybean Oil | q.s |
| Total | 1078.67 |

### Example 19

### Oral (Capsule) Acyclovir Formulation

Acyclovir and SNAC were separately screened through a sieve of pore size of 500 μm (USP standard sieve # 35). Afterwards, predetermined amounts of acyclovir and SNAC were mixed in a mortar and a pestle for 3 minutes. In a beaker, the required amounts of soybean oil, Capryol PGMC and labrasol were mixed until homogeneous. To the powder blend, the solvent was added drop wise while mixing in a mortar and pestle. The final blend was mixed for 3 minutes and later filled into a hard gelatin capsule.

TABLE 19
Components of Acyclovir/SNAC capsule made with
emulsifying solvents.

| Ingredients | mg/capsule | |
|---|---|---|
| Acyclovir | 240 | |
| SNAC | 240 | |
| Soybean oil | 40 | |
| Capryol PGMC | 40 | |
| Labrasol | 20 | |
| Total Weight | 580 | mg |

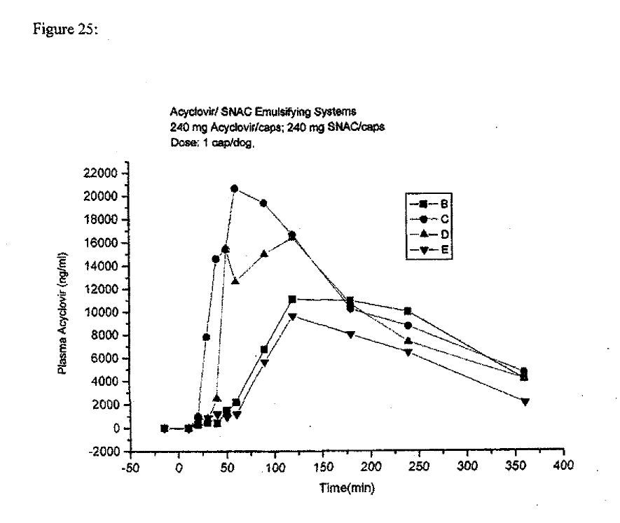

FIG. 25 shows pharmacokinetic profiles of acyclovir capsules formulated with emulsifying solvents. This data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 0 | 0 | 0 | 0 |
| 20 | 292.2 | 1002.4 | 482 | 673.7 |
| 30 | 485.8 | 7845.9 | 842.6 | 875.7 |
| 40 | 439.9 | 14637.9 | 2536.1 | 1245 |
| 50 | 1505.7 | 15459.6 | 15471.6 | 967 |
| 60 | 2198.3 | 20696 | 12679.7 | 1214.5 |
| 90 | 6766 | 19452.3 | 15061.9 | 5692.2 |
| 120 | 11159.1 | 16718.2 | 16488 | 9714.7 |
| 180 | 10988.7 | 10254.1 | 10688.2 | 8049.8 |
| 240 | 10029.4 | 8780.8 | 7436 | 6491.5 |
| 360 | 4192.6 | 4761.5 | 4209.7 | 2149.2 |

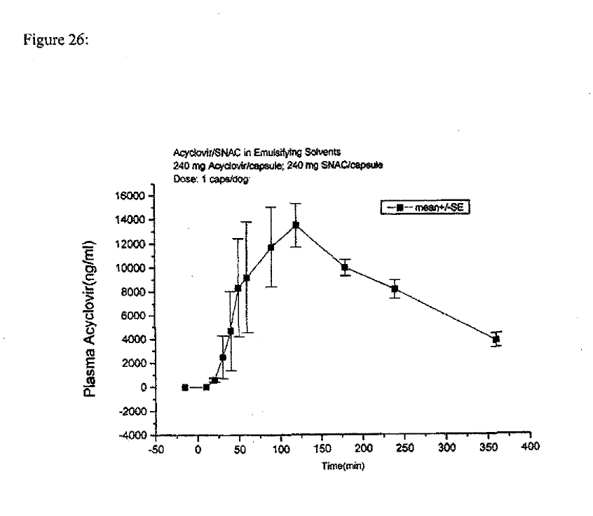

FIG. 26 shows mean plasma acyclovir levels after dosing acyclovir/SNAC capsules formulated with emulsifying solvents. Each data point represents the mean+/−SE (n=4 beagles).

### Example 20

### Oral (Capsules) Acyclovir Formulation

Acyclovir and SNAC were separately screened through a sieve of pore size of 500 μm (USP standard sieve # 35). Afterwards, predetermined amounts (table 20) of acyclovir and SNAC were mixed in a mortar and a pestle for 3 minutes. In a beaker, the required amounts (table 20) of soybean oil, propylene glycol mono caprylate and caprylocapryol polyoxylglycerides were mixed until homogeneous. To the powder blend, the solvent was added drop wise while mixing in a mortar and pestle. The final blend was mixed for 10 minutes using a homogenizer and later filled into a hard gelatin capsule. The dose was filled into 2 capsules.

TABLE 20
Components of acyclovir/SNAC capsule made with
emulsifying solvents. The dose was divided into 2
capsules and dosed in beagles.

| Ingredients | mg/dose | |
|---|---|---|
| Acyclovir | 240 | |
| SNAC | 240 | |
| Soybean | 280 | |
| Propylene glycol mono | 280 | |
| caprylate | | |
| Caprylocaproyl | 140 | |
| polyoxylglycerides | | |
| Total Weight | 1180 | mg |

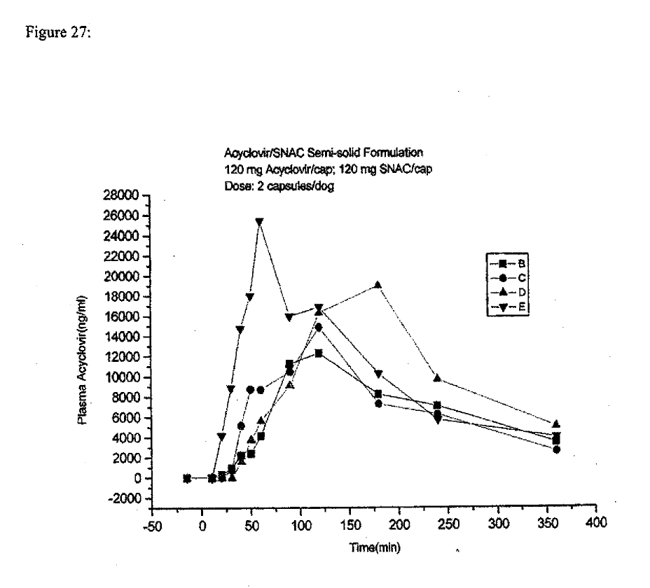

FIG. 27 shows pharmacokinetic profiles of acyclovir/SNAC semisolid formulation made with emulsifying solvents. Each capsule contains 120 mg of acyclovir and 120 mg of SNAC. The dose per beagle is 2 capsules. This data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 0 | 0 | 0 | 0 |
| 20 | 299.3 | 0 | 0 | 4193 |
| 30 | 955.7 | 765.1 | 0 | 8877.2 |
| 40 | 2228.7 | 5175.4 | 1611.3 | 14761.6 |
| 50 | 2399.6 | 8761 | 3770.5 | 18024 |
| 60 | 4173.8 | 8727.8 | 5635.3 | 25444.6 |
| 90 | 11266.6 | 10503.8 | 9141 | 16005.9 |
| 120 | 12326.8 | 14913.5 | 16361.6 | 16931.8 |
| 180 | 8246.7 | 7291.4 | 19046.3 | 10298.9 |
| 240 | 7071.8 | 6241.5 | 9719.2 | 5708.4 |
| 360 | 3458.2 | 2544.5 | 5045.5 | 3980.8 |

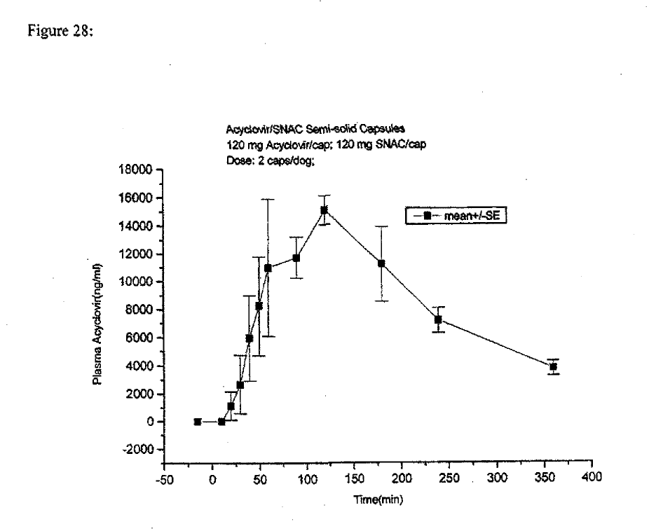

FIG. 28 shows mean levels of acyclovir in beagle plasma after dosing acyclovir/SNAC semi-solid formulation with emulsifying solvents. Each data point represents the mean+/−SE (n=4 beagles).

### Example 21

### Oral (Tablets) Acyclovir Formulation

Tablets were prepared by the process of wet granulation. All ingredients were screened through a sieve of pore size of 500 μm (USP standard sieve #35). The predetermined weight of each component per tablet of the formulation (table 21) was adjusted based on the batch size (number of tablets to be prepared). For instance, the amounts of acyclovir and SNAC needed to prepare 20 tablets are acyclovir 4800 mg and SNAC 4800 mg; accordingly the weights of all other excipients were adjusted based on the batch size.

The required amounts of acyclovir and SNAC were weighed and blended for 3 minutes using a mortar and a pestle. To the blend, the required amounts of potassium aluminum sulfate, povidone and croscarmellose sodium were added. The powder mixture was blended using a mortar and a pestle for 5 minutes. Purified water was added to the powder blend drop wise while mixing the powder blend to obtain wet granules. Wet granules were dried in a vacuum oven (Isotemp Model 282A; Fisher Scientific) at 55° C. for about 8 hours until the moisture level in the granules was less than 5%. The moisture level in the dry granules was measured by solid drop to a Brinkmann 737 Karl Fisher Coulometer. Dry granules were screened through a sieve of pore size 500 μm. Extragranular excipients as shown in table 21: pregelatinized starch, croscarmellose sodium and magnesium stearate were added and blended for 5 minutes. The weight per dose of the final blend of (dry granules and extragranular excipients) was compressed to tablets using a caplet-shaped tool on a single punch tablet press Korsch XL100. The targeted tablet properties are: hardness of 8-10 KP and disintegration time of 6-8 minutes in water at 37° C.

While the invention is not limited to a particular mechanism, Potassium aluminum sulfate is an astringent which may have modified gastric emptying.

TABLE 21
Components of acyclovir/SNAC tablet formulation that was
made with potassium alum sulfate.

| Ingredients | mg/tablet | |
|---|---|---|
| Acyclovir | 240 | |
| SNAC | 240 | |
| Potassium aluminum sulfate | 4.8 | |
| Kollidon 90F | 3.2 | |
| Croscarmellose sodium | 12 | |
| Pregelatinized Starch (extragranular) | 62 | |
| Magnesium stearate (extragranular) | 5 | |
| Croscarmellose sodium (extragranular) | 5 | |
| Total Weight | 572 | mg |

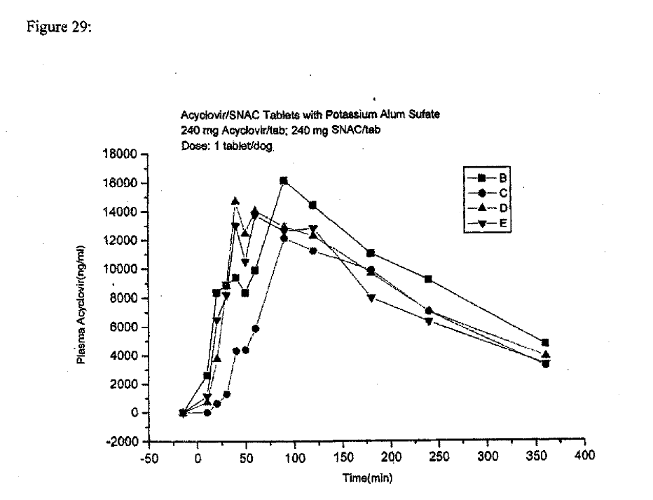

FIG. 29 shows pharmacokinetic profiles of acyclovir/SNAC tablet formulations made with potassium aluminum sulfate. This data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 2631.1 | 0 | 734.9 | 1167.5 |
| 20 | 8372.1 | 650.4 | 3768.9 | 6495.3 |
| 30 | 8874.4 | 1303.3 | 8819 | 8226.2 |
| 40 | 9406.2 | 4332.8 | 14717 | 13053.4 |
| 50 | 8358.1 | 4401.5 | 12481.3 | 10526.6 |
| 60 | 9900.3 | 5905.2 | 14081 | 13759.4 |
| 90 | 16200.2 | 12160.5 | 12962.5 | 12663.4 |
| 120 | 14473.6 | 11259.8 | 12335.5 | 12872.2 |
| 180 | 11062.8 | 9883.3 | 9690.3 | 8007.9 |
| 240 | 9235.6 | 7042.9 | 7066.2 | 6365.1 |
| 360 | 4767.6 | 3244 | 3921.7 | 3368.8 |

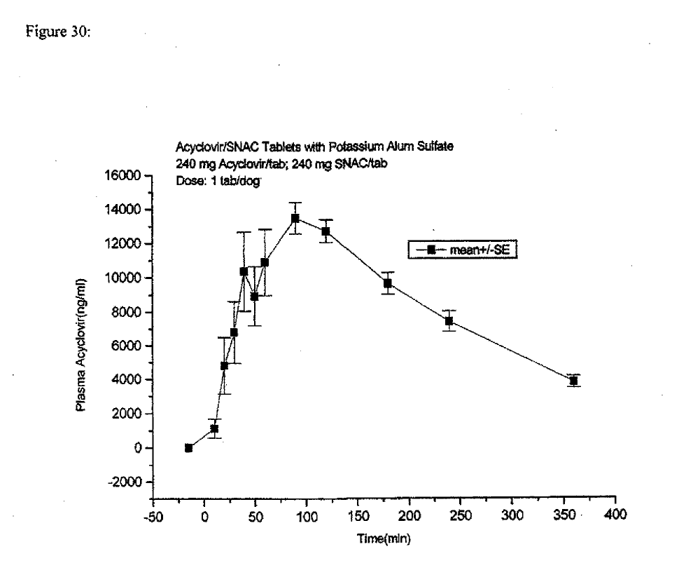

FIG. 30 shows mean levels of acyclovir in beagle plasma after dosing acyclovir/SNAC tablets formulated with potassium alum sulfate Each data point represents the mean+/−SE (n=4 beagles).

### Example 22

### Oral (Tablets) Acyclovir Formulation

The process of Example 21 was repeated except lecithin was used in place of potassium alum sulfate, and an aqueous solution of polysorbate 80 was prepared and used as the granulating solution in place of purified water. To prepare polysorbate 80 solution, 0.5 g of polysorbate was weighed and dissolved in 7.5 g of water by gentle stirring on a magnetic stirrer. Amounts of each ingredient are as shown below in Table 22.

TABLE 22
Components of acyclovir/SNAC tablet formulation that was
made with lecithin and polysorbate 80.

| Ingredients | mg/tablet | |
|---|---|---|
| Acyclovir | 240 | |
| SNAC | 240 | |
| Povidone | 3 | |
| Croscarmellose sodium | 12 | |
| Lecithin | 48 | |
| Polysorbate 80 | 4.8 | |
| Pregelatinized Starch (extragranular) | 65.4 | |
| Magnesium stearate (extragranular) | 5.4 | |
| Croscarmellose sodium (extragranular) | 5.4 | |
| Total Weight | 624 | mg |

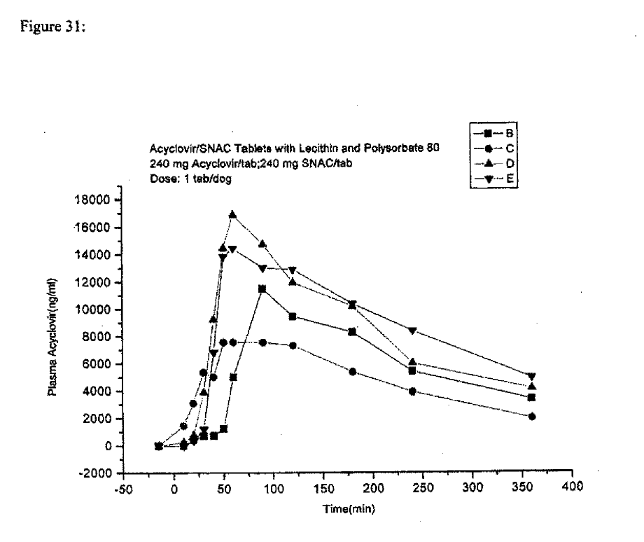

FIG. 31 shows pharmacokinetic profiles of acyclovir/SNAC tablet formulations made with lecithin and polysorbate 80. This data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 0 | 1460.8 | 273.1 | 0 |
| 20 | 434 | 3101.5 | 792.2 | 385.3 |
| 30 | 724.9 | 5364.6 | 3891.9 | 1216.3 |
| 40 | 750.5 | 5037.7 | 9236.9 | 6840.4 |
| 50 | 1240.5 | 7565.7 | 14484.4 | 13852.5 |
| 60 | 5023.5 | 7571.8 | 16903.2 | 14449.2 |
| 90 | 11526.5 | 7558.8 | 14785.8 | 13056.2 |
| 120 | 9482.7 | 7324.6 | 11982.8 | 12934.3 |
| 180 | 8301 | 5371.9 | 10213.3 | 10396.4 |
| 240 | 5422.6 | 3908.1 | 6033.6 | 8416.1 |
| 360 | 3359.6 | 1972.3 | 4145.1 | 4953.4 |

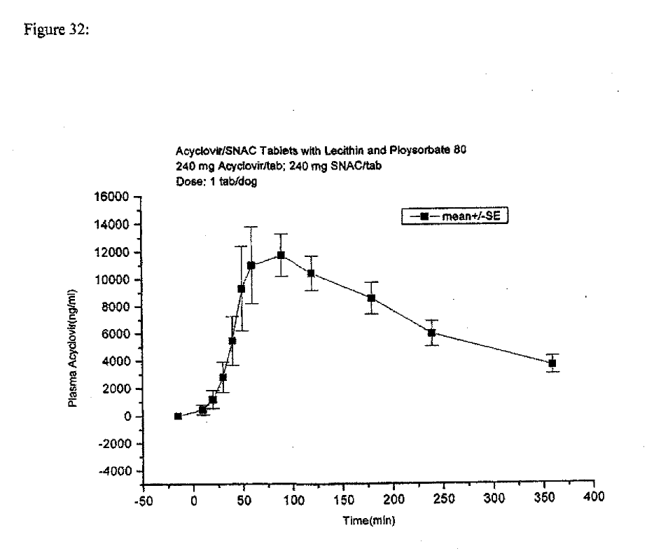

FIG. 32 shows mean levels of acyclovir in beagle plasma after dosing acyclovir/SNAC tablets formulated with lecithin and polysorbate 80. Each data point represents the mean+/−SE (n=4 beagles).

### Example 23

### Oral (Tablets) Acyclovir Formulation

The process of Example 21 was repeated except gelatin and lecithin were used in place of potassium alum sulfate and povidone. Amounts of each ingredient was added as shown in Table 23.

TABLE 23
Components of acyclovir/SNAC tablet formulation that was
made with gelatin and lecithin.

| Ingredients | mg/tablet | |
|---|---|---|
| Acyclovir | 240 | |
| SNAC | 240 | |
| Gelatin | 24 | |
| Croscarmellose sodium | 13 | |
| Lecithin | 48 | |
| Pregelatinized Starch (extragranular) | 67.8 | |
| Magnesium stearate (extragranular) | 5.6 | |
| Croscarmellose sodium (extragranular) | 5.6 | |
| Total Weight | 644 | mg |

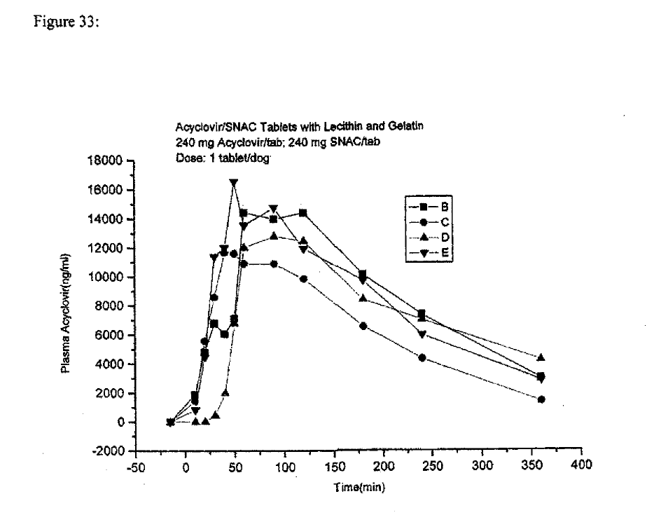

FIG. 33 shows pharmacokinetic profiles of acyclovir/SNAC tablet formulations made with gelatin and lecithin. This data is also shown below.

TABLE 33A

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 1905.7 | 1443.2 | 0 | 839.7 |
| 20 | 4812 | 5572.1 | 0 | 4480.5 |
| 30 | 6785.5 | 8574.8 | 445.7 | 11402.9 |
| 40 | 6055.6 | 11689.8 | 2001.7 | 12010.4 |
| 50 | 7126.9 | 11616.2 | 6783.2 | 16539.8 |
| 60 | 14438.9 | 10908.8 | 12016.8 | 13566.6 |
| 90 | 13990.6 | 10906.9 | 12807.4 | 14775.3 |
| 120 | 14426.2 | 9854 | 12478.4 | 11964.8 |
| 180 | 10138.2 | 6558.3 | 8402.8 | 9722.6 |
| 240 | 7365.8 | 4328.4 | 7022.6 | 5985.3 |
| 360 | 2978.4 | 1376.3 | 4237.4 | 2849.9 |

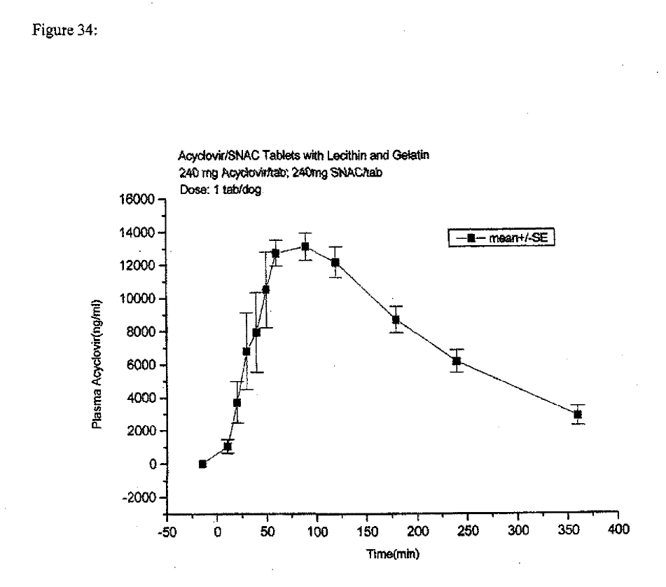

FIG. 34 shows mean levels of acyclovir in beagle plasma after dosing acyclovir/SNAC tablets formulated with gelatin and lecithin. Each data point represents the mean+/−SE (n=4 beagles).

### Example 24

### Oral (Tablets) Acyclovir Formulation

The process of Example 21 was repeated except lecithin was used in place of potassium aluminum sulfate. Each ingredient was added in the amount shown in Table 24.

TABLE 24
Components of acyclovir/SNAC (360 mg/360 mg) tablet
formulation made with lecithin.

| Ingredients | mg/tablet | |
|---|---|---|
| Acyclovir | 360 | |
| SNAC | 360 | |
| Povidone | 4 | |
| Croscarmellose sodium | 18 | |
| Lecithin | 72 | |
| Pregelatinized Starch (extragranular) | 97 | |
| Magnesium stearate (extragranular) | 8 | |
| Croscarmellose sodium (extragranular) | 8 | |
| Total Weight | 927 | mg |

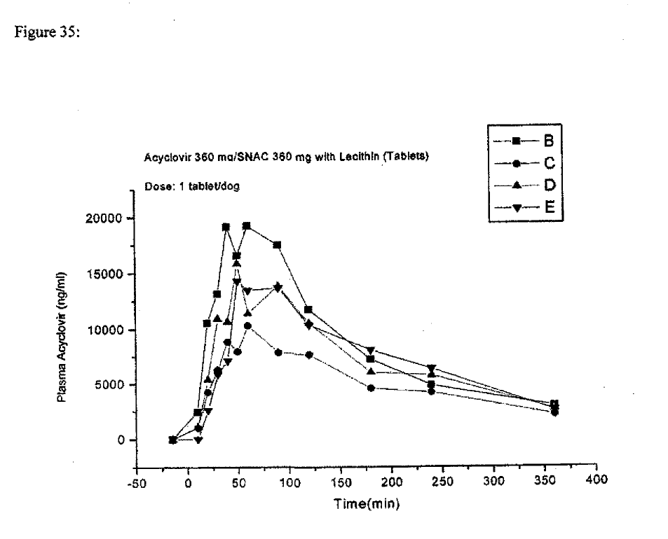

FIG. 35 shows pharmacokinetic profiles of acyclovir/SNAC (360 mg/360 mg) tablet formulations made with lecithin. This data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 2455 | 1010.2 | 1079.1 | 0 |
| 20 | 10576.7 | 4246.4 | 5406.6 | 2603.9 |
| 30 | 13163.3 | 6269.1 | 10953.7 | 5896.9 |
| 40 | 19157.3 | 8848.6 | 10683.5 | 7127.9 |
| 50 | 16594.9 | 7967.7 | 15854.5 | 14308.9 |
| 60 | 19250.4 | 10321.6 | 11432.3 | 13476 |
| 90 | 17516.4 | 7877.6 | 13850.3 | 13683.5 |
| 120 | 11788.2 | 7687.6 | 10571.7 | 10390.8 |
| 180 | 7253.3 | 4586.6 | 6064.6 | 8105.1 |
| 240 | 4857.3 | 4215.8 | 5794.7 | 6392.7 |
| 360 | 2958.3 | 2178.8 | 2672.6 | 2601.3 |

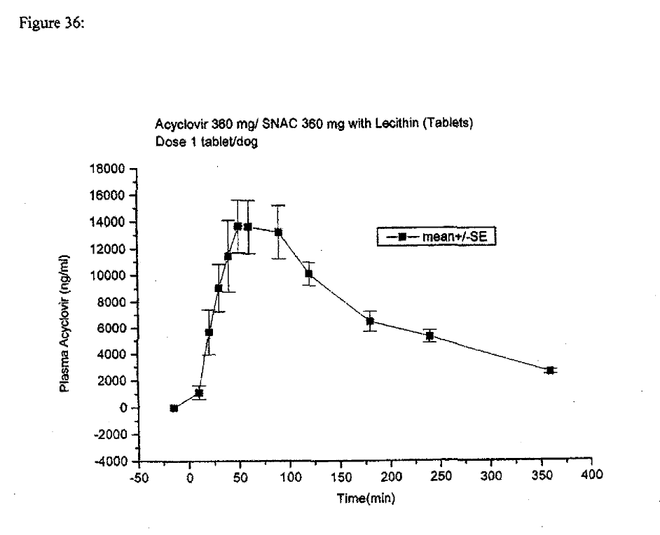

FIG. 36 shows mean levels of acyclovir in beagle plasma after dosing acyclovir/SNAC (360 mg/360 mg) tablets formulated with lecithin. Each data point represents the mean+/−SE (n=4 beagles).

### Example 25

### Oral (Tablets) Acyclovir Formulation

The process of Example 21 was repeated except gelatin and sodium lauryl sulfate were used in place of potassium alum sulfate and povidone. Each ingredient was added in the amount shown in Table 25.

TABLE 25
Components of acyclovir/SNAC (360 mg/360 mg) tablet
formulation made with gelatin and sodium lauryl sulfate.

| Ingredients | mg/tablet | |
|---|---|---|
| Acyclovir | 360 | |
| SNAC | 360 | |
| Gelatin | 72 | |
| Croscarmellose sodium | 18 | |
| Sodium lauryl sulfate | 14 | |
| Pregelatinized Starch (extragranular) | 98.8 | |
| Magnesium stearate (extragranular) | 8.2 | |
| Croscarmellose sodium (extragranular) | 8.2 | |
| Total Weight | 939 | mg |

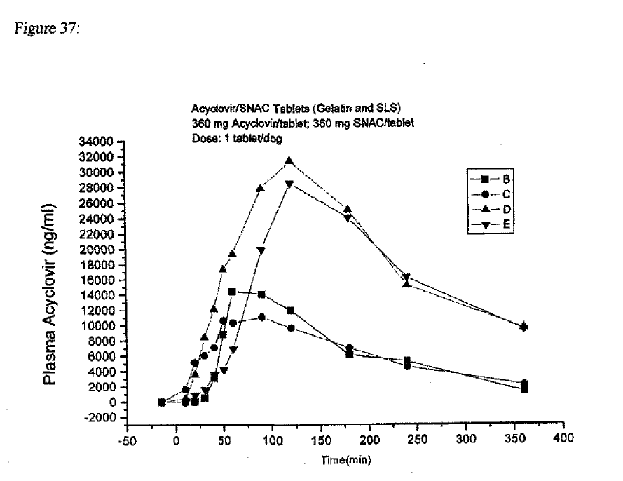

FIG. 37 shows pharmacokinetic profiles of acyclovir/SNAC (360 mg/360 mg) tablet formulations made with gelatin and sodium lauryl sulfate. This data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 0 | 1648.7 | 442.3 | 0 |
| 20 | 0 | 5146.6 | 3565.7 | 876.9 |
| 30 | 504.9 | 6053.9 | 8432.3 | 1630.2 |
| 40 | 3143.2 | 7085.8 | 12116.1 | 3577.7 |
| 50 | 8822.5 | 10589.7 | 17304.7 | 4233.6 |
| 60 | 14421 | 10327.3 | 19331 | 6907.3 |
| 90 | 14080.8 | 11033.5 | 27842.5 | 19906.7 |
| 120 | 12003.1 | 9689.6 | 31405.8 | 28569.5 |
| 180 | 6150.2 | 7054.7 | 25124.7 | 24119.3 |
| 240 | 5307.6 | 4611.1 | 15277.9 | 16301 |
| 360 | 1329.7 | 2203.1 | 9655.9 | 9472.3 |

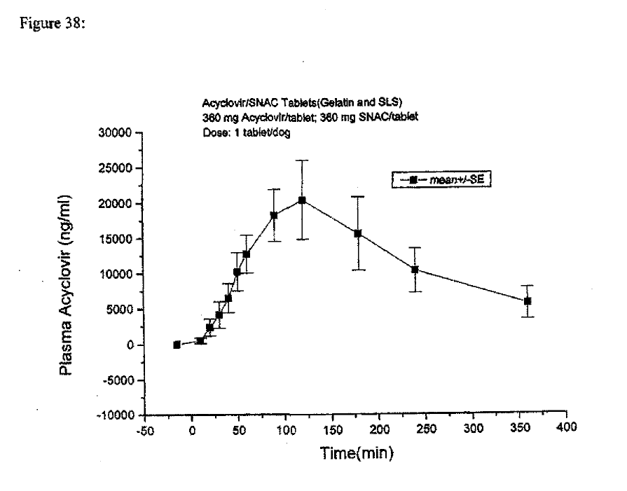

FIG. 38 shows mean levels of acyclovir in beagle plasma after dosing acyclovir/SNAC (360 mg/360 mg) tablets formulated with gelatin and sodium lauryl sulfate. Each data point represents the mean+/−SE (n=4 beagles).

### Example 26

### Oral (Tablets) Acyclovir Formulation

The process of Example 21 was repeated except potassium alum sulfate was not used, and an aqueous solution of polysorbate 80 was prepared and used as the granulating solution in place of purified water. To prepare polysorbate 80 solution, 0.3 g of polysorbate was weighed and dissolved in 4 g of water by gentle stirring on a magnetic stirrer. Each ingredient was added in the amount shown in Table 26.

TABLE 26
Components of acyclovir/SNAC (360 mg/360 mg) tablet
formulation made with Polysorbate 80.

| Ingredients | mg/tablet | |
|---|---|---|
| Acyclovir | 360 | |
| SNAC | 360 | |
| Povidone | 7.2 | |
| Croscarmellose sodium | 18 | |
| Polysorbate 80 | 15 | |
| Pregelatinized Starch (extragranular) | 91 | |
| Magnesium stearate (extragranular) | 7.6 | |
| Croscarmellose sodium (extragranular) | 7.6 | |
| Total Weight | 866 | mg |

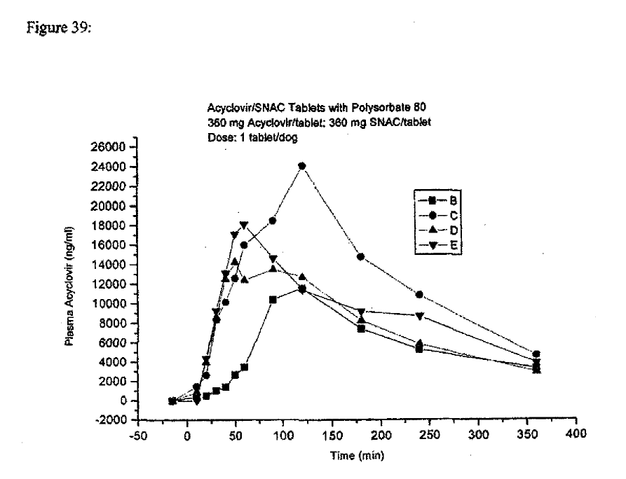

FIG. 39 shows pharmacokinetic profiles of acyclovir/SNAC (360 mg/360 mg) tablet formulations made with polysorbate 80. This data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 409.5 | 1472.6 | 896.3 | 0 |
| 20 | 529.2 | 2644 | 4002.9 | 4390.4 |
| 30 | 1061.2 | 8392.2 | 8625.3 | 9279.4 |
| 40 | 1435.2 | 10165.9 | 12533.2 | 13126.7 |
| 50 | 2662.6 | 12594.7 | 14276.3 | 17108.4 |
| 60 | 3461.1 | 16007.9 | 12440.5 | 18146.4 |
| 90 | 10437 | 18521.2 | 13520.8 | 14662.2 |
| 120 | 11586 | 24118.3 | 12754.7 | 11453.7 |
| 180 | 7450.2 | 14796 | 8322.9 | 9241.9 |
| 240 | 5289.6 | 10865.1 | 5864.3 | 8740.4 |
| 360 | 3340.5 | 4698.6 | 3004.4 | 3933.3 |

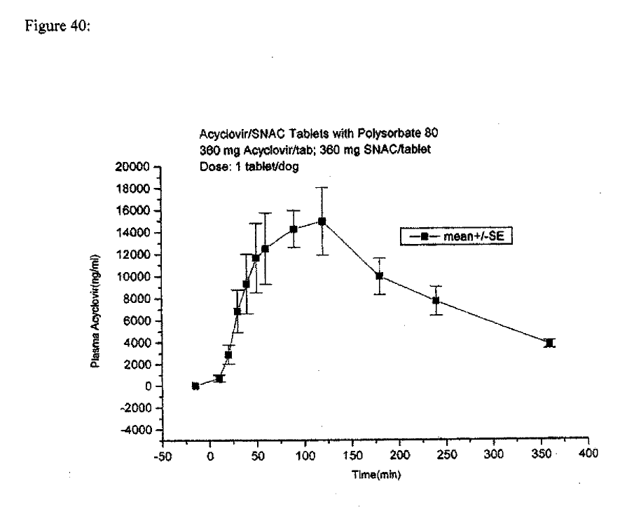

FIG. 40 shows mean levels of acyclovir in beagle plasma after dosing acyclovir/SNAC (360 mg/360 mg) tablets formulated with polysorbate 80. Each data point represents the mean+/−SE (n=4 beagles).

### Example 27

### Oral (Capsules) Acyclovir Formulation

The process of Example 15 was repeated except that unmicronized SNAC was used instead of micronized SNAC, croscarmellose sodium was used in place of soybean oil and Gelucire 50/13 was added in addition to Gelucire 44/14. Amounts of each ingredient are shown in Table 27.

TABLE 27
Components of acyclovir/SNAC (360 mg/360 mg) capsule
formulation made with gelatin, sodium lauryl sulfate and Gelucire.

| Ingredients | mg/tablet | |
|---|---|---|
| Acyclovir | 360 | |
| SNAC | 360 | |
| Gelatin | 72 | |
| Croscarmellose sodium | 18 | |
| Gelucire 44/14 | 98 | |
| Gelucire 50/13 | 42 | |
| Total Weight | 950 | mg |

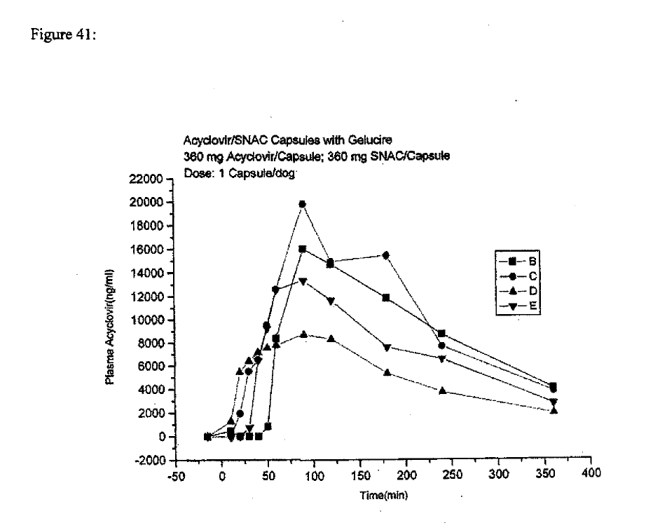

FIG. 41 shows pharmacokinetic profiles of acyclovir/SNAC (360 mg/360 mg) capsules formulated with Gelucire. This data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 459.9 | 0 | 1283.4 | 0 |
| 20 | 0 | 1951.4 | 5489 | 0 |
| 30 | 0 | 5539.2 | 6442.5 | 746.2 |
| 40 | 0 | 6552.4 | 7192 | 6502 |
| 50 | 845.5 | 9567.2 | 7603.4 | 9257 |
| 60 | 8404.7 | 12595.4 | 7807 | 12563.4 |
| 90 | 16065.8 | 19842.9 | 8770.2 | 13372.3 |
| 120 | 14747.5 | 14974.9 | 8369.8 | 11684.9 |
| 180 | 11870.6 | 15461.2 | 5371.9 | 7642.6 |
| 240 | 8735.1 | 7721.5 | 3744.2 | 6604 |
| 360 | 4058.8 | 3813.7 | 1950.3 | 2767.6 |

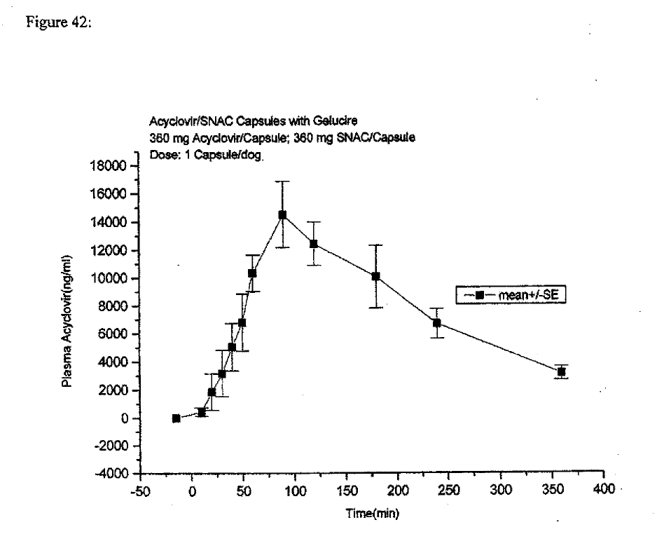

FIG. 42 shows mean levels of acyclovir/SNAC (360 mg/360 mg) capsule formulation in a beagle model. Each data point represents the mean+/−SE (n=4 beagles).

### Example 28

### Oral (Tablets) Acyclovir Formulation

The process of Example 15 was repeated except that unmicronized SNAC was used instead of micronized SNAC, sodium lauryl sulfate was added and a mixture of PEG 300 and Capmul was used as the granulating fluid instead of purified water. The amounts of each ingredient were added as shown in Table 28.

TABLE 28
Components of acyclovir/SNAC (300 mg/300 mg) tablet
formulation made with PEG 300 and Capmul.

| Ingredients | mg/tablet | |
|---|---|---|
| Acyclovir | 300 | |
| SNAC | 300 | |
| Gelatin | 60 | |
| PEG 300 | 14 | |
| Capmul | 33 | |
| Croscarmellose sodium | 18 | |
| Sodium Lauryl Sulfate | 18 | |
| Croscarmellose sodium (extragranular) | 7.43 | |
| Pregelatinized starch | 89 | |
| Magnesium stearate | 7.43 | |
| Total Weight | 950 | mg |

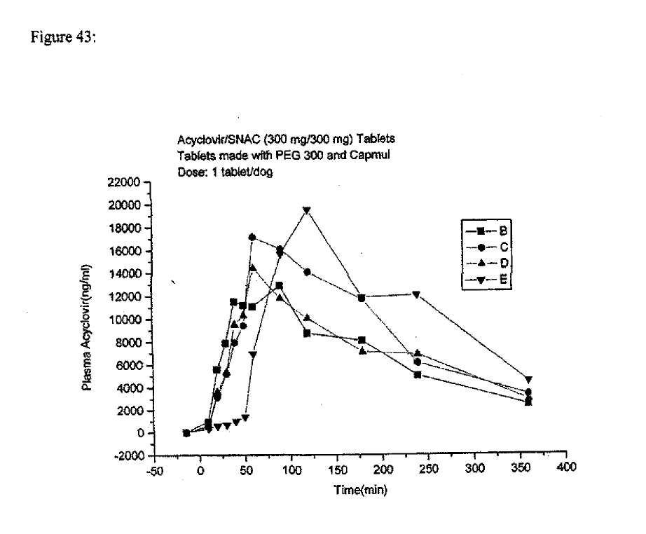

FIG. 43 shows pharmacokinetic profiles of acyclovir/SNAC (300 mg/300 mg) tablet formulation made with PEG 300 and Capmul. This data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 956.2 | 590.8 | 519.4 | 345.7 |
| 20 | 5577.5 | 3171.8 | 3611.6 | 609.5 |
| 30 | 7869.5 | 5190.2 | 5352.8 | 683.7 |
| 40 | 11490.7 | 7916.1 | 9492.8 | 975.4 |
| 50 | 11177 | 9408.3 | 10315.7 | 1404.9 |
| 60 | 11046.9 | 17138.8 | 14399.1 | 6952.6 |
| 90 | 12884.2 | 16094.3 | 11778.9 | 15643.2 |
| 120 | 8756.6 | 14068.5 | 10069.5 | 19562 |
| 180 | 8112 | 11683.2 | 7132 | 11873.9 |
| 240 | 5011.2 | 6149.5 | 6898.1 | 12044.4 |
| 360 | 2440.3 | 3329.6 | 2889.3 | 4494.8 |

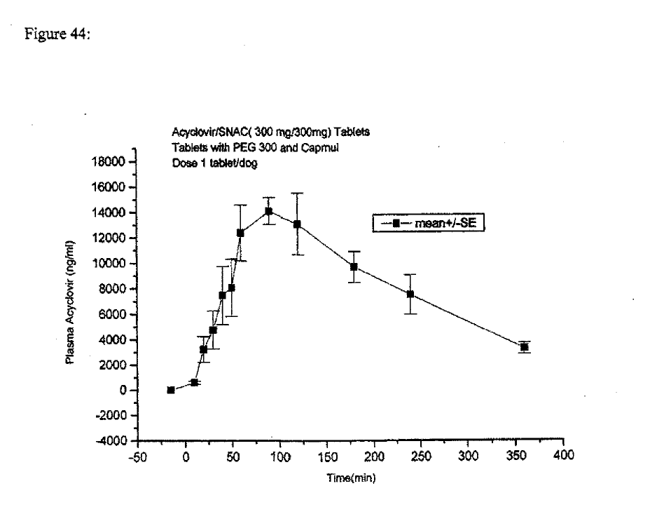

FIG. 44 shows mean levels of acyclovir/SNAC (300 mg/300 mg) tablet formulation made with PEG 300 and Capmul. Each data point represents the mean+/−SE (n=4 beagles).

### Example 29

### Oral (Tablets) Acyclovir Formulation—CONTROL

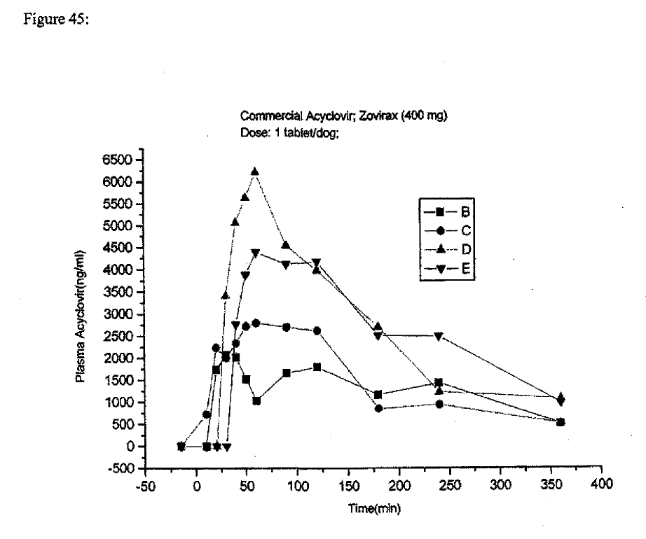

FIG. 45 shows pharmacokinetic profiles of Commercial acyclovir (400 mg) tablets (ZOVIRAX®) in a beagle model. This data is also shown below.

| | |
|---|---|
| Time | |
| (min.) | Four (4) Beagles were Dosed (ng/ml) |

| | | | | |
|---|---|---|---|---|
| −15 | 0 | 0 | 0 | 0 |
| 10 | 0 | 716.4 | 0 | 0 |
| 20 | 1731.9 | 2231.4 | 0 | 0 |
| 30 | 2066.1 | 2002.6 | 3407.4 | 0 |
| 40 | 2010.9 | 2332.1 | 5064.7 | 2764.5 |
| 50 | 1516.9 | 2714.8 | 5628.4 | 3882.2 |
| 60 | 1032.7 | 2792 | 6210.3 | 4389.3 |
| 90 | 1653.4 | 2697.4 | 4539.7 | 4119 |
| 120 | 1793.9 | 2627 | 3989.3 | 4182.8 |
| 180 | 1164.1 | 849.1 | 2707.9 | 2513.2 |
| 240 | 1428.4 | 935.2 | 1227.7 | 2493.7 |
| 360 | 499.2 | 505.6 | 1070.8 | 976.1 |

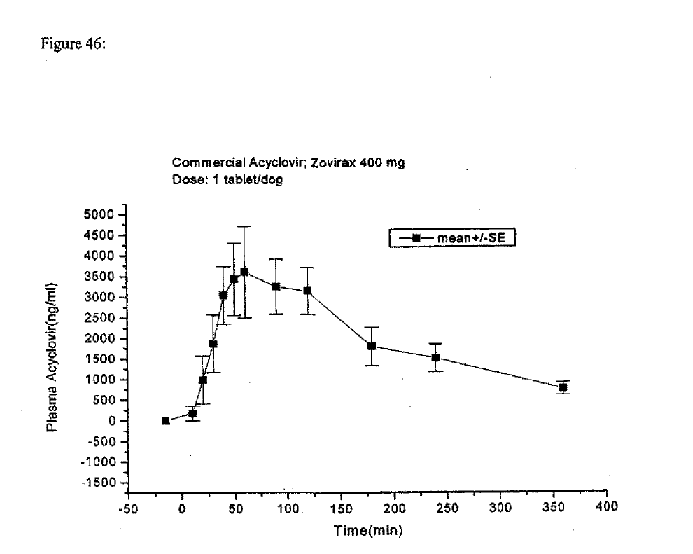

FIG. 46 shows mean levels of acyclovir in beagle plasma after dosing commercial acyclovir (400 mg) tablets (ZOVIRAX®) in a beagle model. Each data point represents the mean+/−SE (n=4 beagles)

### Example 30

### Oral (Tablet) Acyclovir Clinical Formulations

A study of the comparative bioavailability of acyclovir, after administration of Zovirax®, Valtrex® or a acyclovir/SNAC formulation shown below in Table 29 (240 mg/240 mg) in 12 healthy male subjects under fasting conditions was performed.

In order to reduce variability, a randomized, crossover (3×3), latin square design with three treatment groups, three periods, and three sequences was selected to study the pharmacokinetic parameters of the study medication and two reference medications on each subject. No concomitant medication was given to the subjects during the course of the clinical stage of this study.

Acyclovir pharmacokinetic parameters were obtained by the non-compartmental analysis of plasma concentration-time data. Relative bioavailability and dose normalized peak exposure ratios were assessed to compare study medication with reference medications. A washout interval of 7 days was chosen to prevent pharmacological/pharmacokinetics treatment interactions.

The treatment arms were as follows:

Treatment A: (acyclovir/SNAC, 240 mg/240 mg) oral tablets shown in Table 29.

Treatment B: ZOVIRAX® (acyclovir) 800 mg oral tablets.

Treatment C: VALTREX® (valacyclovir) 1000 mg oral caplets.

The tablets of treatment arm A were prepared as described in Example 8, except Aerosil was added to the formulation.

TABLE 29
Oral tablets administered in treatment arm A.

| Ingredients | mg/tablet |
|---|---|
| Acyclovir | 240 |
| SNAC | 240 |
| Kollidon 90F (Povidone k90), | 3.0 |
| (intragranular, 0.5%) | |
| *Ac-Di-Sol (Croscarmellose | 12.0 |
| sodium) (Intragranular, 2.0%) | |
| *Ac-Di-Sol (Croscarmellose | 6.0 |
| sodium) (Extragranular, 1.0%) | |
| Pre-gelatinized Starch (Starch 1500 ®) | q.s |
| Magnesium Stearate (1% w/w) | 6.0 |
| Aerosil | 12 |
| Total | 600 |

The following demographic data was obtained from the subjects: age, height, weight, and body mass index (BMI).

TABLE 30
Demographic Parameters

| | Age | Weight | Height | BMI |
|---|---|---|---|---|
| Subject | (years) | (kg) | (cm) | (kg/m2) |
| 1 | 20 | 65 | 173 | 21.7 |
| 2 | 40 | 81.8 | 174 | 27.0 |
| 3 | 34 | 68.8 | 174 | 22.7 |
| 4 | 49 | 67.5 | 178 | 21.3 |
| 5 | 22 | 72.4 | 176 | 23.4 |
| 6 | 40 | 77 | 169 | 27.0 |
| 7 | 22 | 63.8 | 169 | 22.3 |
| 8 | 28 | 72.6 | 173 | 24.3 |
| 9 | 40 | 60.1 | 168 | 21.3 |
| 10 | 20 | 74 | 170 | 25.6 |
| 11 | 25 | 62.7 | 165 | 23.0 |
| 12 | 31 | 58.8 | 161 | 22.7 |
| Mean | 30.9 | 68.7 | 170.8 | 23.5 |
| SD | 9.7 | 7.0 | 4.8 | 2.0 |

Sample Handling: For taking of the blood samples, an intravenous catheter was placed, which after obtaining each sample had a device placed with a syringe with sodium heparin (0.6 units for samples with the intervals between each less than 2 hrs and 0.8 units for those samples with intervals between each over 2 hrs), to bathe the inside of the catheter and keep it from clogging. This device with the blood that has been extracted to cleanse the catheter was discarded before obtaining the next blood sample, which was used for the study.

6-mL blood samples were taken with sterile plastic syringes and were placed in tubes containing heparin as an anti-coagulant. Blood samples were drawn through a catheter as first choice or by venipuncture. The blood samples were refrigerated (2 to 8° C.) for a maximum of 60 minutes before centrifuge.

Centrifugation was performed at 3000±200 rpm, at 4±2° C. during 15 minutes. The plasma that was obtained was separated from the cellular fraction, it was placed in two cryotubes (first and second series) and was stored immediately at −40±5° C. until their shipment to Fundación Liomont A. C.

6-mL venous blood samples were taken by means of a catheter or venipuncture at 0 hour (pre-dose) and at 5, 10, 15, 20, 25, 30, 35, 40, 45, 50 minutes and 1.0, 1.5, 2.0, 3.0, 4.0, 6.0, 8.0, 12.0 and 24.0 hours after the dose. The plasma obtained was collected as described above. Intervals between dosing and blood sample collection between subjects were kept so that the scheduled times for these activities are the same for all subjects. The exact time of sample collection was recorded in the appropriate CRF and initialed by the person who collected.

The samples were stored in an ultra-freezer at ±70° C.±5° C. until their analysis.

Plasma acyclovir levels were determined using a HPLC method developed in house with acetaminophen used as internal standard. 0.2 mL of plasma plus 0.1 mL of internal standard (acetaminophen, 200 μg/mL) and 0.1 mL of 7% perchloric acid were shaken in a test tube and centrifuged at 3500 rpm for 10 minutes at room temperature. Supernatant was passed through a regenerated cellulose syringe filter (pore size 0.45 microns) and injected into the chromatographic system. Separation of compounds was performed in a 15-cm×4.6-mm inside diameter of 5 μm particle size, Zorbax® SB-C8, and eluted with a mobile phase consisting of 0.02 M perchloric acid, pH 2.0±0.1. The column temperature was 25° C. Flow rate was maintained constant at 1.5 mL/min, and detection was carried out by fluorescence at 260 nm and 375 nm of excitation and emission wavelength, respectively.

The non-compartmental analysis module of WinNonlin® Professional Edition Version 5.0 (Pharsight Corporation), pharmacokinetic model 200, extra-vascular input for the oral data, was used for pharmacokinetic parameter calculations.

The following results were obtained:

TABLE 31
Average pharmacokinetic parameters

| | Tmax | Cmax | AUC₀₋₂₄ | AUC_{0-∞} | | | MRT₀₋₂₄ | MRT_{0-∞} | AUMC_{0-∞} |
|---|---|---|---|---|---|---|---|---|---|
| Treatment | (h)) | (ng/mL) | (hr * ng/mL) | (hr * ng/mL) | Kel (h⁻¹) | t_{1/2} (h) | (h) | (h) | (h * h * ng/mL) |
| A | 1.444 | 533.226 | 2178.763 | 2625.114 | 0.235 | 3.597 | 3.432 | 5.391 | 14716.276 |
| B | 1.903 | 815.284 | 3509.640 | 4017.701 | 0.201 | 3.638 | 3.907 | 5.508 | 22463.723 |
| C | 1.992 | 4973.740 | 17209.537 | 18080.238 | 0.236 | 3.085 | 3.699 | 4.342 | 78561.057 |

(A) acyclovir/SNAC,
(B) Zovirax ® (acyclovir) 800 mg tablets,
(C) Valtrex ® (valacyclovir) 1000 mg caplets

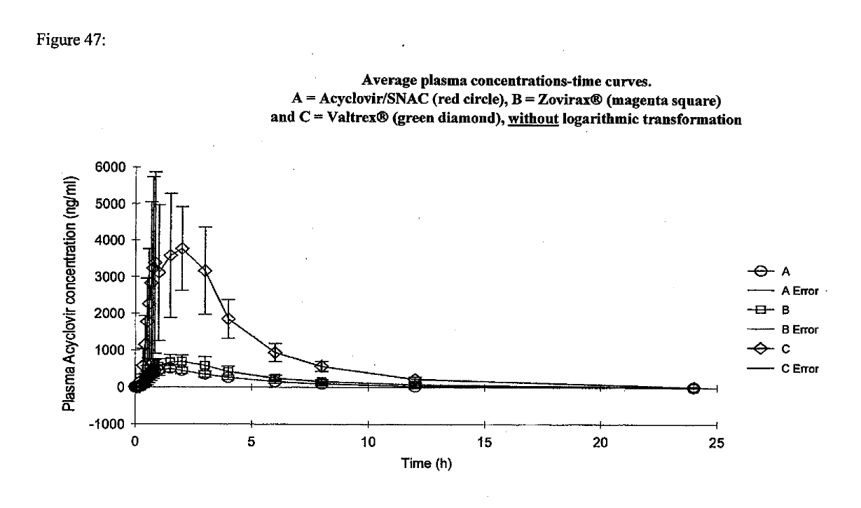

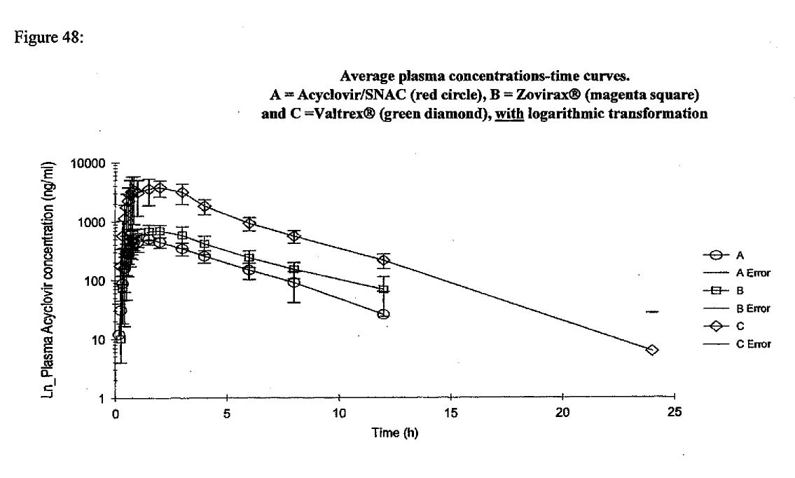

FIG. 47 shows Average plasma concentrations-time curves. A=acyclovir/SNAC (circle), B=Zovirax® (square) and C Valtrex® (diamond), without logarithmic transformation
FIG. 48 shows Average plasma concentrations-time curves. A=acyclovir/SNAC (circle), B=Zovirax® (square) and C=Valtrex® (diamond), with logarithmic transformation

Pharmacokinetic parameters (logarithmic transformation of Cₘₐₓ, AUC₀₋₂₄, and AUC_{0-∞}) estimated for the three treatments were compared by using 95% confidence intervals of the mean differences between treatments:

Acyclovir/SNAC—Zovirax® (A-B)

Acyclovir/SNAC—Valtrex® (A-C)

Valacyclovir—Acyclovir (C-B)

It was considered as a statistically significant difference, a difference of means in which the zero value (μ₁−μ₂=0) was not included within the corresponding 95% confidence interval.

Analysis of variance (ANOVA) was performed on these variables (Cmax, AUC0-24, and

AUC0-∞) with treatment, period, and sequence in the model; subject within sequence was included as a random variable. The results of the ANOVA indicated that among all of the factors, only treatment was significant, whereas sequence and period resulted to be not-significant (α=0.05).

Residual variance from ANOVA was used to construct the 95% confidence intervals. The mean treatment differences (log-transformed) and their corresponding 95% confidence intervals and the probability values (p-values) are shown in Table 32.

All differences resulted significant because the 95% confidence intervals do not include the zero value and their corresponding p-values are less than 0.05 (α=0.05).

TABLE 32
95% confidence intervals for log-transformed data

| Parameter | | | Lower | Upper | |
|---|---|---|---|---|---|
| (Ln) | Comparison | Difference | CI 95% | CI 95% | p-value |
| Cmax | A-B | −0.42 | −0.62 | −0.22 | <0.0001 |
| (ng/mL) | A-C | −2.19 | −2.39 | −1.99 | <0.0001 |
| | B-C | −1.78 | −1.98 | −1.57 | <0.0001 |
| AUC₀₋₂₄ | A-B | −0.48 | −0.63 | −0.33 | <0.0001 |
| (ng · hr/mL) | A-C | −2.07 | −2.22 | −1.92 | <0.0001 |
| | B-C | −1.59 | −1.74 | −1.44 | <0.0001 |
| AUC_{0-∞} | A-B | −0.43 | −0.57 | −0.29 | <0.0001 |
| (ng · hr/mL) | A-C | −1.93 | −2.07 | −1.8 | <0.0001 |
| | B-C | −1.5 | −1.64 | −1.36 | <0.0001 |

The relative bioavailability (F) of acyclovir from the study medication (treatment A: Acyclovir/SNAC 240 mg/240 mg) with respect to acyclovir 800 mg tablets (treatment B) and valacyclovir 1000 mg caplets (treatment C) were estimated by using the following equation:
${f}_{C max} = \frac{C {max}_{TEST}}{{DOSE}_{TEST}} \times \frac{{DOSE}_{REF}}{C {max}_{REF}} F = \frac{{AUC}_{0 - {∞}_{TEST}}}{{DOSE}_{TEST}} \times \frac{{DOSE}_{REF}}{{AUC}_{0 - {∞}_{REF}}}$
Where TEST = to treatment A and REF = to treatment B or treatment C.

TABLE 33
Relative bioavailability and dose normalized peak exposure ratio

| Treatment | f | F |
|---|---|---|
| A/B | 2.18 | 2.18 |
| A/C | 0.279 | 0.377 |

(A) acyclovir/SNAC 240 mg/240 mg tablets,
(B) ZOVIRAX ® (acyclovir) 800 mg tablets,
(C) VALTREX ® (valacyclovir) 1000 mg caplets

Table 33 presents the values obtained for relative bioavailability (F) and dose normalized peak exposure ratio (f), parameters that compare dose normalized Cₘₐₓ and AUC_{0--∞} values of study medication with those of reference medications. The results showed that the exposure to acyclovir from the study medication (acyclovir/SNAC 240 mg/240 mg) was about twice that of acyclovir 800 mg tablets and about one third that of acyclovir from valacyclovir 1000 mg caplets.

All differences (Cₘₐₓ, AUC₀₋₂₄ and AUC_{0--∞}) between mean treatments were significant (α=0.05).

VALTREX® (valacyclovir) caplets had the highest Cₘₐₓ, AUC₀₋₂₄ and AUC_{0--∞}, values, followed by ZOVIRAX® (acyclovir) 800 mg tablets, and lastly the study medication acyclovir/SNAC (240 mg/240 mg) tablets had the lowest Cₘₐₓ, AUC₀₋₂₄ and AUC_{0--∞} values.

Although the study medication (acyclovir/SNAC 240 mg/240 mg) had the lowest values for the studied pharmacokinetic parameters, when both indexes of exposure, Cₘₐₓ and AUC_{0-∞}, were dose normalized and compared to references, acyclovir exposure resulted to be about twice that of acyclovir 800 mg tablets and approximately half that of acyclovir from VALTREX®. These results demonstrate that acyclovir absorption can be increased by SNAC.

The above-mentioned patents, applications, test methods, and publications are hereby incorporated by reference in their entirety.

Many variations of the present invention will suggest themselves to those skilled in the art in light of the above detailed description. All such obvious variations are within the fully intended scope of the appended claims.

1. A solid pharmaceutical composition comprising:
(a) SNAC;
(b) acyclovir; and
(c) a disintegrant,
wherein the SNAC and acyclovir are at least substantially dissolved within an hour of contact with an aqueous medium.

2. The pharmaceutical composition of claim 1, wherein the SNAC and acyclovir are completely dissolved in the aqueous medium.

3. The pharmaceutical composition of claim 1, wherein the aqueous medium is simulated gastric fluid, simulated intestinal fluid, or both.

4. The pharmaceutical composition of claim 1, wherein the aqueous medium is simulated gastric fluid.

5. The pharmaceutical composition of claim 1, wherein the aqueous medium is simulated intestinal fluid.

6. The pharmaceutical composition of claim 1, wherein the aqueous medium is gastric fluid, intestinal fluid, or both.

7. The pharmaceutical composition of claim 1, wherein the aqueous medium is gastric fluid.

8. The pharmaceutical composition of claim 1, wherein the aqueous medium is intestinal fluid.

9. The pharmaceutical composition of claim 1, wherein the weight ratio of SNAC to acyclovir is from about 0.75:1 to about 6:1.

10. The pharmaceutical composition of claim 9, wherein the weight ratio of SNAC to acyclovir is from about 0.9:1 to about 4:1.

11. The pharmaceutical composition of claim 10, wherein the weight ratio of SNAC to acyclovir is about 1:1.

12. The pharmaceutical composition of claim 1, wherein the pharmaceutical composition comprises from about 200 to about 1500 mg of acyclovir.

13. The pharmaceutical composition of claim 12, wherein the pharmaceutical composition comprises from about 240 to about 500 mg of acyclovir.

14. The pharmaceutical composition of claim 12, wherein the pharmaceutical composition comprises from about 240 to about 1000 mg of SNAC.

15. The pharmaceutical composition of claim 12, wherein the pharmaceutical composition comprises from about 480 to about 1000 mg of acyclovir.

16. The pharmaceutical composition of claim 15, wherein the pharmaceutical composition comprises from about 480 to about 2000 mg of SNAC.

17. The pharmaceutical composition of claim 1, wherein the disintegrant is selected from cross-linked N-vinyl-2-pyrrolidone (“CLPVP”), sodium starch glycolate, polacrilin potassium, sodium alginate, microcystalline or microtine cellulose, methyl cellulose, hydroxypropylcellulose, carboxymethyl cellulose sodium, and croscarmellose sodium and a combination of any of the foregoing.

18. The pharmaceutical composition of claim 17, wherein the disintegrant is croscarmellose sodium.

19. The pharmaceutical composition of claim 1, further comprising (d) one or more wetting agents.

20. The pharmaceutical composition of claim 19, wherein the wetting agent is selected from polyethylene glycol, sodium lauryl sulfate, mixtures of glycerol and polyethylene glycol, esters of long-fatty acids, mixtures of monoglycerides and diglycerides of capyrilic and capric acid in glycerol, polypropylene glycol monocaprylate, soyabean oil, propylene glycol mono caprylate, caprylocaproyl polyoxylglycerides, and polysorbate 80 and any combination of any of the foregoing.

21. The pharmaceutical composition of claim 1, further comprising:
(e) a binder; and
(f) a lubricant.

22. The pharmaceutical composition of claim 21, wherein the binder is selected from methyl cellulose, microcrystalline or microtine cellulose, hydroxypropylcellulose, hydroxypropyl methylcellulose, carboxymethylcellulose, povidone, polyvinyl alcohol, gelatin, cassien, glycerine, sorbitol, mannitol, sucrose, lactose, fructose, starch, corn starch, pregelatinized starch, amylose, dextrose, amylodextrin, maltodextrin, cyclodextin, gums and any combination of any of the foregoing.

23. The pharmaceutical composition of claim 22, wherein the binder is povidone.

24. The pharmaceutical composition of claim 22, wherein the binder is gelatin.

25. The pharmaceutical composition of claim 22, wherein the binder is a combination of povidone and pregelatinized starch.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)
